(19) United States
(12) Patent Application Publication
(10) Pub. No.: US 2020/0254037 A1
(43) Pub. Date: Aug. 13, 2020

(54) TREATMENT OF TRIPLE NEGATIVE BREAST CANCER OR COLORECTAL CANCER WITH LIVER METASTASES WITH AN ANTI PD-L1 ANTIBODY AND AN ONCOLYTIC VIRUS

(71) Applicants: AMGEN INC., Thousand Oaks, CA (US); GENENTECH, INC., South San Francisco, CA (US)

(72) Inventors: Jennifer Lorraine Gansert, Simi Valley, CA (US); Sumita Shankar Bhatta, Thousand Oaks, CA (US); Joseph Paul Woodard, JR., South El Monte, CA (US); Edward Namserk Cha, South El Monte, CA (US)

(21) Appl. No.: 16/637,600

(22) PCT Filed: Aug. 6, 2018
(86) PCT No.: PCT/US18/45328

§ 371 Date: Feb. 7, 2020

Related U.S. Application Data
(60) Provisional application No. 62/542,046, filed on Aug. 7, 2017.

(51) Int. Cl. A61K 35/768; A61K 39/395; A61K 9/00; A61P 35/00

(52) U.S. Cl. CPC A61K 35/768; A61K 39/3955; A61K 2039/505; A61P 35/00; A61K 9/0019

(57) ABSTRACT

Provided herein are methods of treating a subject with triple negative breast cancer or colorectal cancer. In exemplary embodiments, the method comprises administering to the subject a combination of an oncolytic virus, such as talimogene laherparepvec, and an anti-PD-L1 antibody, such as atezolizumab. In exemplary aspects, the oncolytic virus is administered to the subject at an initial dose followed by a second dose, wherein the initial dose is lower than the second dose. In exemplary aspects, the oncolytic virus is intrahepatically administered to the subject.

### CROSS REFERENCE TO RELATED APPLICATIONS

The benefit under 35 U.S.C. § 119(e) of U.S. Provisional Patent Application No. 62/542,046, filed on Aug. 7, 2017, is hereby claimed, and the disclosure thereof is hereby incorporated by reference herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 23 kilobyte ACII (Text) file named “51358A_Seqlisting.txt”; created on Jul. 23, 2018.

### BACKGROUND

The incidence rate of breast cancer varies worldwide and the mortality rate is between 10 and 20 per 100,000 women across most global regions (Youlden et al, 2012). Annually in the United States, approximately 232,000 women are diagnosed with, and 40,290 women die from, breast cancer according to the Surveillance, Epidemiology, and End Results (SEER) Program. Triple negative tumors account for about 15% of all invasive breast cancers (Foulkes et al, 2010). Among metastatic triple-negative cases, the first distant site is lung (40%), brain (30%), liver (20%), and bone (10%) (Foulkes et al, 2010). With subsequent metastases, the liver will be diagnosed as a metastatic site in up to 50% of women with metastatic triple negative breast cancer (Lin et al, 2008). The estimated 5-year survival rate for metastatic triple negative breast cancer is approximately 22% according to the SEER database. Incidence of triple negative breast cancer is increased in patients with germline breast cancer susceptibility gene 1 (BRCA1) mutations and African ancestry. Triple negative breast cancers are generally aggressive tumors with a high rate of distant metastases and worse disease-specific survival than other breast cancer subtypes (Dent et al, 2007; Haffty et al, 2006). Tumors with the triple negative phenotype have specific features that are potential therapeutic targets (e.g., they show an impaired deoxyribonucleic acid (DNA) repair mechanism and increased expression of basal-associated and proliferation associated markers).

There is significant heterogeneity within triple negative breast cancer. A study analyzing gene expression profiling identified 6 subtypes, one of which was an immunomodulatory subtype enriched for genes involved in immune cell processes including immune cell signaling, cytokine signaling, antigen processing and presentation, and signaling through core immune signal transduction pathways (Lehmann et al, 2011). In addition, the clinical importance of tumor immune infiltrates has been an emerging area of research in triple negative breast cancer, where an increased number of immune infiltrates seems to predict both response to chemotherapy and improved survival in the neoadjuvant setting and is a prognostic factor in the adjuvant setting (Adams et al, 2014; Dieci et al, 2014; Ono et al, 2012).

Data indicate that PD-1/PD-L1 pathway blockade has clinical activity in patients with metastatic triple negative breast cancer. PD-L1 is expressed in approximately 20% of patients with triple negative breast cancer and treatment with anti PD-1 and anti PD-L1 agents (e.g., pembrolizumab, atezolizumab, or nivolumab) is currently under investigation in several ongoing trials.

Colorectal cancer incidence rates vary worldwide, with rates per 100,000 among males ranging from 4 in India to 59 in the Czech Republic (Center et al, 2009). Annually in the United States, approximately 132,700 people are diagnosed with, and 49,700 people die from, colorectal cancer (SEER). The proportion of patients with synchronous liver metastases at initial diagnosis is about 15% and the 5-year cumulative metachronous liver metastasis rate has been reported to be 4% for stage I tumors, 13% for stage II, and 30% for stage III (Manfredi et al, 2006). For 3 of 4 cases at diagnosis of liver metastasis, the liver is the only metastatic site (Manfredi et al, 2006). Evidence of activity of PD-1 based therapy in colorectal cancer is provided by Brahmer et al, 2012, which describes a phase 1 study of nivolumab that included 39 patients with various solid tumors,1/14 patients with metastatic MSI-high colorectal cancer had a durable complete response. Also, Le et al., 2015, describes a phase 2 study with 41 patients in which administration of pembrolizumab monotherapy resulted in objective response of 40% of patients with MSI phenotype and at least SD in 78% compared with 0% and 11% in patients with MSS colorectal cancer. Despite these advances, the exact mechanism of checkpoint inhibitors in colorectal cancer is still unknown.

While immune therapies are valid approaches in cancer therapy, such therapies appear to be effective only in a proportion of patients with cancer. Consequently, researchers are exploring different approaches to improve therapeutic efficacy. Thus, there remains a need for improved methods of treating subjects with triple negative breast cancer and colorectal cancer.

### SUMMARY

Provided herein are methods of treating a subject with triple negative breast cancer or colorectal cancer. In exemplary embodiments, the method comprises administering to the subject a combination of an oncolytic virus and an anti-PD-L1 antibody. In exemplary aspects, the oncolytic virus is an agent that increases tumor-specific immune activation and the anti-PD-L1 antibody blocks an inhibitory T cell checkpoint. Without being bound to a particular theory, this combination produces a greater antitumor activity than either agent alone in both triple negative breast cancer and colorectal cancer. In exemplary aspects, the oncolytic virus is talimogene laherparepvec and the anti-PD-L1 antibody is atezolizumab. Without being bound to a particular theory, talimogene laherparepvec augments dendritic cell-mediated tumor antigen presentation through local expression of GM-CSF and local antigen release by direct tumor lysis, and atezolizumab regulates the actions of PD-L1 and prevents T cell exhaustion in peripheral tissues. In exemplary embodiments, the oncolytic virus is administered to the subject at an initial dose followed by a second dose. In exemplary instances, the initial dose is lower than the second dose. In exemplary aspects, the method comprises administering to the subject a combination of talimogene laherparepvec and atezolizumab, wherein talimogene laherparepvec is administered to the subject at an initial dose followed by a second dose, and the initial dose is lower than the second dose.

Also provided herein are methods of treating a subject with triple negative breast cancer with liver metastases or with colorectal cancer with liver metastases. In exemplary aspects, the method comprises administering to the subject a combination of an oncolytic virus and an anti-PD-L1 antibody, wherein the oncolytic virus is intrahepatically administered to the subject. In exemplary aspects, the oncolytic virus is an agent that increases tumor-specific immune activation and the anti-PD-L1 antibody blocks inhibitory T cell checkpoints. In exemplary aspects, the oncolytic virus is talimogene laherparepvec and the anti-PD-L1 antibody is atezolizumab. In exemplary aspects, the method comprises administering to the subject a combination of talimogene laherparepvec and atezolizumab, wherein talimogene laherparepvec is intrahepatically administered to the subject.

Further provided are methods of treating a subject with triple negative breast cancer or colorectal cancer metastases. In exemplary embodiments, the method comprises administering to the subject a combination of an oncolytic virus and an anti-PD-L1 antibody, wherein the oncolytic virus is administered to the subject at an initial dose followed by a second dose, wherein the initial dose is lower than the second dose. In exemplary aspects, the oncolytic virus is an agent that increases tumor-specific immune activation and the anti-PD-L1 antibody blocks inhibitory T cell checkpoints. In exemplary aspects, the oncolytic virus is talimogene laherparepvec and the anti-PD-L1 antibody is atezolizumab. In exemplary aspects, the method comprises administering to the subject a combination of talimogene laherparepvec and atezolizumab, wherein talimogene laherparepvec is administered to the subject at an initial dose followed by a second dose, wherein the initial dose is lower than the second dose. In exemplary embodiments, the method comprises administering to the subject a combination of an oncolytic virus and an anti-PD-L1 antibody, wherein the oncolytic virus is intrahepatically administered to the subject. In exemplary aspects, the oncolytic virus is an agent that increases tumor-specific immune activation and the anti-PD-L1 antibody blocks inhibitory T cell checkpoints. In exemplary aspects, the oncolytic virus is talimogene laherparepvec and the anti-PD-L1 antibody is atezolizumab. In exemplary aspects, the method comprises administering to the subject a combination of talimogene laherparepvec and atezolizumab, wherein talimogene laherparepvec is intrahepatically administered to the subject.

### BRIEF DESCRIPTION OF THE DRAWINGS

FIG. 1 is an illustration of the study design and treatment schema described in Example 1.

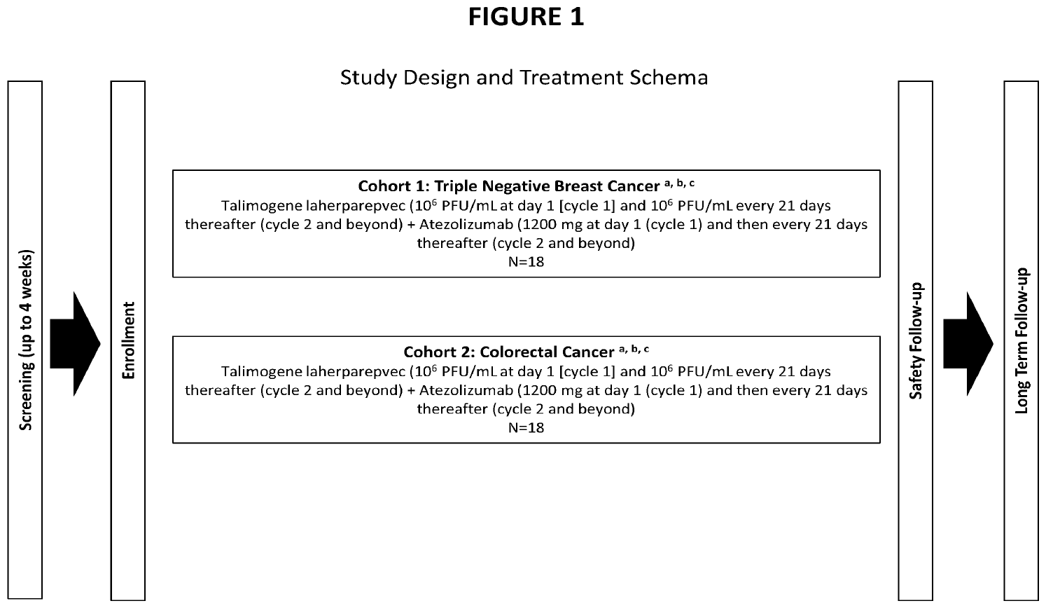

### DETAILED DESCRIPTION

Provided herein are methods of treating a subject with triple negative breast cancer or colorectal cancer. In exemplary embodiments, the method comprises administering to the subject a combination of an oncolytic virus and an anti-PD-L1 antibody.

Oncolytic Virus

Oncolytic immunotherapy is an emerging treatment modality which uses replication competent oncolytic viruses that will selectively infect and damage cancerous tissues without causing harm to normal tissues. Each oncolytic virus has a specific cellular tropism that determines which tissues are preferentially infected and genetic engineering can occur to make them cancer specific while rendering them nonpathogenic to normal host cells (Russell et al, 2014). Ongoing studies are using a variety of engineered viruses not limited to herpes simplex virus (HSV), vaccinia, and reovirus.

In exemplary aspects, the oncolytic virus is derived from a vaccinia virus. In exemplary instances, the oncolytic virus is a modified vaccinia virus with a disrupted thymidine kinase (tk) gene, an insertion of human granulocyte-macrophage colony-stimulating factor (GM-CSF), an insertion of β-galactosidase, or a combination thereof. In exemplary aspects, the oncolytic virus is JX-594 (pexastimogene devacirepvec (Pexa-Vec)). See, e.g., Park et at, 2008

In exemplary aspects, the oncolytic virus is derived from a herpes simplex virus 1 (HSV1) or herpes simplex 2 (HSV2) strain, or from a derivative thereof, preferably HSV1. Derivatives include inter-type recombinants containing DNA from HSV1 and HSV2 strains. Such inter-type recombinants are described in the art, for example in Thompson et al., (1998) Virus Genes 1(3); 275286, and Meignier et al., (1998) J. Infect. Dis.159; 602614.

Herpes simplex virus strains may be derived from clinical isolates. Such strains are isolated from infected individuals, such as those with recurrent cold sores. Clinical isolates may be screened for a desired ability or characteristic such as enhanced replication in tumor and/or other cells in vitro and/or in vivo in comparison to standard laboratory strains, as described in U.S. Pat. Nos. 7,063,835 and 7,223,593, each of which are incorporated by reference in their entirety. In one embodiment the herpes simplex virus is a clinical isolate from a recurrent cold sore.

Herpes simplex virus 1 virus strains include, but are not limited to, strain JS1, strain 17+, strain F, strain KOS, and strain Patton.

Herpes simplex viruses may be modified, for example, as compared to their precursor strain, such that the modified virus lacks one or more functional viral genes. As used herein, “lacking a functional” viral gene means that the gene(s) is partially or completely deleted, replaced, rearranged, or otherwise altered in the herpes simplex genome such that a functional viral protein can no longer be expressed from that gene by the herpes simplex virus.

Examples of HSV genes that can be modified include virulence genes encoding proteins such as ICP34.5 (γ34.5). ICP34.5 acts as a virulence factor during HSV infection, limits replication in non-dividing cells and renders the virus non-pathogenic. Another HSV gene that can be modified is the gene encoding ICP47. ICP47 down-regulates major histocompatibility complex (MHC) class I expression on the surface of infected host cells and MHC Class I binding to transporter associated with antigen presentation (TAP). Such actions block antigenic peptide transport in the endoplasmic reticulum and loading of MHC class I molecules. Another HSV gene that can be modified is ICP6, the large subunit of ribonucleotide reductase, involved in nucleotide metabolism and viral DNA synthesis in non-dividing cells but not in dividing cells. Thymidine kinase, responsible for phosphorylating acyclovir to acyclovir-monophosphate, virion trans-activator protein vmw65, glycoprotein H, vhs, ICP43, and immediate early genes encoding ICP4, ICP27, ICP22 and/or ICP0, may be modified as well (in addition or alternative to the genes referenced above).

Modifications may also be made to alter the timing of expression of herpes simplex virus genes. For example, Us11 can be expressed as an early gene by placing the Us11 gene under the Us12 promoter (Mulvey et al. (1999) J Virology, 73:4, 3375-3385; US Patent Number 5,824,318; Mohr & Gluzman (1996) EMBO 15: 4759-4766).

Examples of modified herpes simplex viruses include, but are not limited to, the Seprehvi™ (HSV1716) strain 17+ of herpes simplex virus type 1 having a deletion of 759 bp located within each copy of the BamHI s fragment (0 to 0-02 and 0-81 to 0.83 map units) of the long repeat region of the HSV genome, removing one complete copy of the 18 bp DR˜ element of the ‘a’ sequence and terminates 1105 bp upstream of the 5′ end of immediate early (1E) gene 1 (see MacLean et al., (1991) Journal of General Virology 79:631-639).

Another example is G207, an oncolytic HSV-1 derived from wild-type HSV-1 strain F having deletions in both copies of the major determinant of HSV neurovirulence, the ICP 34.5 gene, and an inactivating insertion of the E. coli lacZ gene in UL39, which encodes the infected-cell protein 6 (ICP6) (see Mineta et al. (1995) Nat Med.1:938-943).

Another example is OrienX010, a herpes simplex virus with deletion of both copies of Û34.5 and the ICP47 genes as well as an interruption of the ICP6 gene and insertion of the human GM-CSF gene (see Liu et al., (2013) World Journal of Gastroenterology 19(31):5138-5143).

Another example is NV1020, a herpes simplex virus with the joint region of the long (L) and short (S) regions deleted, including one copy of ICP34.5, UL24, and UL56.34,35. The deleted region was replaced with a fragment of HSV-2 US DNA (US2, US3 (PK), gJ, and gG) (see Todo, et al. (2001) Proc Natl Acad Sci USA.98:6396-6401).

M032 is a herpes simplex virus with deletion of both copies of the ICP34.5 genes and insertion of interleukin 12 (see Cassady and Ness Parker, (2010) The Open Virology Journal 4:103-108).

Another example is talimogene laherparepvec, derived from a clinical strain, HSV-1 strain JS1, deposited at the European collection of cell cultures (ECAAC) under accession number 01010209. In talimogene laherparepvec, the HSV-1 viral genes encoding ICP34.5 and ICP47 have been functionally deleted. Functional deletion of ICP47 leads to earlier expression of US11, a gene that promotes virus growth in tumor cells without decreasing tumor selectivity. The coding sequence for human GM-CSF, has been inserted into the viral genome at the former ICP34.5 sites (see Liu et al., Gene Ther 10: 292-303, 2003).

ImmunoVEX HSV2 is a herpes simplex virus (HSV-2) having functional deletions of the genes encoding vhs, ICP47, ICP34.5, UL43 and US5.

OncoVEX^{GALV/CD}, is also derived from HSV-1 strain JS1 with the genes encoding ICP34.5 and ICP47 having been functionally deleted and the gene encoding cytosine deaminase and gibbon ape leukaemia fusogenic glycoprotein inserted into the viral genome in place of the ICP34.5 genes.

Additional examples of modified herpes simplex viruses include NSC-733972, HF-10, BV-2711, JX-594, Myb34.5, AE-618, Brainwel™, and Heapwel™.

Herpes virus strains and how to make such strains are also described in U.S. Pat. Nos. 5,824,318; 6,764,675; 6,770,274; 7,063,835; 7,223,593; 7,749,745; 7,744,899; 8,273,568; 8,420,071; and 8,470,577; WIPO Publication Numbers WO199600007; WO199639841; WO199907394; WO200054795; WO2006002394; and WO201306795; Chinese Patent Numbers CN128303, CN10230334 and CN 10230335; Varghese and Rabkin, (2002) Cancer Gene Therapy 9:967-97, and Cassady and Ness Parker, (2010) The Open Virology Journal 4:103-108.

The herpes simplex viruses of the disclosure may also comprise one or more heterologous genes. “Heterologous gene” refers to a gene to be introduced to the genome of a virus, wherein that gene is not normally found in the virus' genome or is a homolog of a gene expressed in the virus from a different species which has a different nucleic acid sequence and can act via a different biochemical mechanism. The heterologous genes may encode one or more proteins, for example, a cytotoxin, an immunomodulatory protein (i.e., a protein that either enhances or suppresses a host immune response to an antigen), a tumor antigen, prodrug activator, a tumor suppressor, a prodrug converting enzyme, proteins capable of causing cell to cell fusion, a TAP inhibitorantisense RNA molecule, or a ribozyme. Examples of immunomodulatory proteins include, for example, cytokines. Cytokines include interleukins, such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20; Interferon (IFN)-α, IFNβ, or IFN-γy-, tumor necrosis factor alpha (TNFα), CD40L, granulocyte macrophage colony stimulating factor (GM-CSF), macrophage colony stimulating factor (M-CSF), and granulocyte colony stimulating factor (G-CSF), chemokines (such as neutrophil activating protein (NAP), macrophage chemoattractant and activating factor (MCAF), RANTES, and macrophage inflammatory peptides MIP-1a and MIP-1b), complement components and their receptors, immune system accessory molecules (e.g., B7.1 and B7.2), adhesion molecules (e.g., ICAM-1, 2, and 3), and adhesion receptor molecules. Tumor antigens include the E6 and E7 antigens of human papillomavirus, EBV-derived proteins, mucins, such as MUC1, melanoma tyrosinase, and MZ2-E. Pro-drug activators include nitroeductase and cytochrome p450, tumour suppressors include p53. a prodrug converting enzymes include cytosine deaminase. Proteins capable of causing cell to cell fusion include gibbon ape leukaemia fusogenic glycoprotein. TAP inhibitors include the bovine herpesvirus (BHV) UL49.5 polypeptide. Antisense RNA molecules that can be used to block expression of a cellular or pathogen mRNA. RNA molecules that can be a ribozyme (e.g., a hammerhead or a hairpin-based ribozyme) designed either to repair a defective cellular RNA, or to destroy an undesired cellular or pathogen-encoded RNA.

Also included is insertion of multiple viral genes into the herpes simplex genome, such as insertion of one or more copies of the gene encoding viral protein Us11.

Anti-PD-L1 Antibodies

A variety of suitable anti-PD-L1 antibodies are contemplated for the methods of the present disclosure. Described herein are several exemplary anti-PD-L1 antibodies that can be used in the methods provided herein.

In any of the embodiments herein, the anti-PD-L1 antibodies can bind to a human PD-L1, for example a human PD-L1 as shown in UniProtKB/Swiss-Prot Accession No.Q9NZQ7.1 1, or a variant thereof. In exemplary embodiments, the anti-PD-L1 antibodies can bind to an epitope of human PD-L1. By “epitope” is meant the region of or within PD-L1 which is bound by the anti-PD-L1 antibody. In some embodiments, the epitope is a linear epitope. “Linear epitope” refers to the region of or within the PD-L1 which is bound by the anti-PD-L1 antibody and which region is composed of contiguous amino acids of the amino acid sequence of the PD-L1. The amino acids of a linear epitope are adjacent to each other in the primary structure of the PD-L1. Accordingly, a linear epitope is a fragment or portion of the amino acid sequence of the antigen, i.e., PD-L1.

In other exemplary embodiments, the epitope is a conformational or structural epitope. By “conformational epitope” or “structural epitope” is meant an epitope which is composed of amino acids which are located in close proximity to one another only when the PD-L1 is in its properly folded state. Unlike linear epitopes, the amino acids of a conformational or structural epitope are not adjacent to each other in the primary structure (i e , amino acid sequence) of the PD-L1. A conformational or structural epitope is not made of contiguous amino acids of the amino acid sequence of the antigen (PD-L1).

In exemplary embodiments, the anti-PD-L1 antibodies bind to PD-L1 in a non-covalent and reversible manner In exemplary embodiments, the binding strength of anti-PD-L1 antibody to PD-L1 may be described in terms of its affinity, a measure of the strength of interaction between the binding site of the anti-PD-L1 antibody and the epitope. In exemplary aspects, the anti-PD-L1 antibody has high-affinity for PD-L1 and thus will bind a greater amount of PD-L1 in a shorter period of time than low-affinity anti-PD-L1 antibodies. In exemplary aspects, the anti-PD-L1 antibody has an equilibrium association constant, KA, which is at least 10⁵ mol⁻¹, at least 10⁶ mol⁻¹, at least 10⁷ mol⁻¹, at least 10⁸ mol⁻¹, at least 10⁹ mol⁻¹, or at least 10¹⁰ mol⁻¹. In exemplary aspects, the anti-PD-L1 antibody exhibits high affinity for PD-L1 in human blood (e.g., 10⁹ mol⁻⁻¹ to 10¹² mol⁻⁻¹).

In exemplary embodiments, the binding strength of the anti-PD-L1 antibody to PD-L1 may be described in terms of its sensitivity. KD is the equilibrium dissociation constant, a ratio of k_{off}/kₒₙ, between the anti-PD-L1 antibody and PD-L1. KD and KA are inversely related. The KD value relates to the concentration of the anti-PD-L1 antibody (the amount of anti-PD-L1 antibody needed for a particular experiment) and so the lower the KD value (lower concentration) the higher the affinity of the anti-PD-L1 antibody. In exemplary aspects, the binding strength of the anti-PD-L1 antibody to PD-L1 may be described in terms of KD. In exemplary aspects, the KD of the anti-PD-L1 antibody is about 10⁻¹ M or less, about 10⁻² M or less, about 10⁻³ M or less, about 10⁻⁴ M or less, about 10⁻⁵ M or less, or about 10⁻⁶ M or less. In exemplary aspects, the KD of the anti-PD-L1 antibody is micromolar, nanomolar, picomolar or femtomolar. In exemplary aspects, the KD of the anti-PD-L1 antibody is within a range of about 10⁻⁻⁴ M to 10⁻⁶ M or 10⁻⁷ M to 10⁻⁹ M or 10⁻¹⁰ M to 10⁻¹² M or 10⁻¹³ M to 10⁻¹⁵ M. In exemplary aspects, the KD of the anti-PD-L1 antibody is within a range of about 10⁻¹² M to about 10⁻⁸ M. In exemplary aspects, the KD of the anti-PD-L1 antibody is within a range of about 10⁻¹¹ M to about 10⁻⁹ M.

Avidity gives a measure of the overall strength of an antibody-antigen complex. It is dependent on three major parameters: affinity of the anti-PD-L1 antibody for the epitope, valency of both the anti-PD-L1 antibody and PD-L1, and structural arrangement of the parts that interact. The greater an anti-PD-L1 antibody's valency (number of antigen binding sites), the greater the amount of antigen (PD-L1) it can bind. In exemplary aspects, the anti-PD-L1 antibody has a strong avidity for PD-L1. In exemplary aspects, the anti-PD-L1 antibody is multivalent. In exemplary aspects, the anti-PD-L1 antibody is bivalent.

In some embodiments, the anti-PD-L1 antibody is capable of inhibiting binding between PD-L1 and PD-1 and/or between PD-L1 and B7-1. The inhibition provided by the anti-PD-L1 antibody may not be a 100% or complete inhibition or abrogation of the binding interaction between PD-L1 and PD-1 and/or between PD-L1 and B7-1. Rather, there are varying degrees of inhibition of which one of ordinary skill in the art recognizes as having a potential benefit or therapeutic effect. In this respect, the anti-PD-L1 antibody may inhibit the binding interaction between a PD-L1 and PD-1 and/or between PD-L1 and B7-1 to any amount or level. In exemplary embodiments, the anti-PD-L1 antibody provides at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition) of the binding between a PD-L1 and PD-1 and/or between PD-L1 and B7-1. In some embodiments, the anti-PD-L1 antibody completely abrogates the binding interaction between the PD-L1 and PD-1 and/or between PD-L1 and B7-1, such that no binding complexes between PD-L1 and PD-1 and/or between PD-L1 and B7-1 are detectable in a sample obtained from a subject, as measured by, for example, immunoprecipitation, Western blotting, immunohistochemistry, and the like.

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody may be a monoclonal antibody or a polyclonal antibody. In some embodiments, the antibody comprises a sequence that is substantially similar to a naturally-occurring antibody produced by a mammal, e g , mouse, rabbit, goat, horse, chicken, hamster, human, and the like. In this regard, the antibody may be considered as a mammalian antibody, e.g., a mouse antibody, rabbit antibody, goat antibody, horse antibody, chicken antibody, hamster antibody, human antibody, and the like. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects of the present disclosure, the method comprises use of an antigen-binding fragment of an anti-PD-L1 antibody (i.e., antigen-binding antibody fragment, antigen-binding fragment, antigen-binding portion), in place of an anti-PD-L1 antibody. In exemplary instances, the antigen-binding antibody fragment is a Fab fragment or a F(ab′)₂ fragment.

The architecture of antibodies has been exploited to create a growing range of alternative antibody formats that spans a molecular-weight range of at least about 12-150 kDa and has a valency (n) range from monomeric (n=1), to dimeric (n=2) and to trimeric (n=3) to tetrameric (n=4) and potentially higher; such alternative antibody formats are referred to herein as “antibody protein products”.

Antibody protein products include those based on antibody fragments, e.g., scFvs, Fabs and VHH/VH (discussed below), which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary embodiments, the methods of the present disclosure comprise use of an antibody protein product, in place of or in addition to an anti-PD-L1 antibody. In exemplary aspects, the antibody protein product comprises, consists essentially of, or consists of any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody; BsIgG; appended IgG; BsAb fragment; bispecific fusion protein; and BsAb conjugate.

The antibody protein product can be in monomeric form, or polymeric, oligomeric, or multimeric form. In certain embodiments in which the antibody protein product comprises two or more distinct antigen binding regions fragments, the antibody protein product is considered bispecific, trispecific, or multi-specific, or bivalent, trivalent, or multivalent, depending on the number of distinct epitopes that are recognized and bound by the antibody protein product.

In some embodiments, the antigen-binding antibody fragment or antibody protein product is selected from the group consisting of Fab, Fab′-SH, Fv, scFv, and (Fab′)₂ fragments.

Examples of anti-PD-L1 antibodies useful in the methods of the present disclosure and methods of making them are described in WIPO Patent Publication Number WO 2010/077634 and U.S. Pat. No. 8,217,149, both of which are incorporated herein by reference.

In some embodiments, the anti-PD-L1 antibody is atezolizumab (CAS Registry Number: 1422185-06-5). Atezolizumab (Genentech), also known as MPDL3280A, is an anti-PD-L1 antibody. Atezolizumab is a humanized immunoglobulin (Ig) G1 monoclonal antibody. It has been engineered to have a single amino acid substitution that leads to elimination Fc-effector function and to be a non-glycosylated antibody with minimal binding to Fc receptors.

Atezolizumab comprises:

(a) an CDR-H1, CDR-H2, and CDR-H3 sequence of GFTFSDSWIH (SEQ ID NO:2), AWISPYGGSTYYADSVKG (SEQ ID NO: 3) and RHWPGGFDY (SEQ ID NO:4), respectively, and

(b) an CDR-L1, CDR-L2, and CDR-L3 sequence of RASQDVSTAVA (SEQ ID NO:5), SASFLYS (SEQ ID NO:6) and QQYLYHPAT (SEQ ID NO:7), respectively.

Atezolizumab comprises a heavy chain and a light chain sequence, wherein:

(a) the heavy chain variable region sequence
comprises the amino acid sequence:
(SEQ ID NO: 8)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,
and
(b) the light chain variable region sequence
comprises the amino acid sequence:
(SEQ ID NO: 9)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPAT
FGQGTKVEIKR.

Atezolizumab comprises a heavy chain and a light chain sequence, wherein:

(a) the heavy chain comprises the amino acid
sequence:
(SEQ ID NO: 10)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSLGYSLSSVVTVPSSSLGTQTYI
CNVNHKPSNTKVDKKCEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALYHNHYTQKSLSLSPG,
and
(b) the light chain comprises the amino acid
sequence:
(SEQ ID NO: 11)
DIQMTQSPSSLSASVGDRVTITCRASQSVSTAVAWYQQKPGKAPKLLIYS
ASFLYSGVPSRFSGSGSGTDFTLTISSLQPEDFATYYCQQYLHYHPATFG
QGTKVEIKRTVAASPVFIFPPSDEQLKSGTASVVCLLNNFYPREAKVQSK
VDNALQSGNSQESVTEQDSKDSTYSLSSTLTLSKADYEKHKVYACEVTHQ
GLSSPVTKSFNRGEC.

In some embodiments, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer). Avelumab comprises a heavy chain and a light chain sequence, wherein:

(a) the heavy chain comprises the amino acid
sequence:
(SEQ ID NO: 12)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG,
and
(b) the light chain comprises the amino acid
sequence:
(SEQ ID NO: 13)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVLGQPKANPTVTLFPPSSEELQSNKATLVCLISDFYPGAVTV
AWKADGSPVKAGVETTKPSKQSNNKYAASSYLSLTPEQWKSHRSYSCQVT
HEGSTVEKTVAPTECS.

In some embodiments, the anti-PD-L1 antibody comprises the six CDR sequences from the heavy chain and light chain comprising the amino acid sequences of SEQ ID NOs: 2-4 and SEQ ID NOs: 5-7 (e.g., the three heavy chain CDRs from SEQ ID NO: 10 and the three light chain CDRs from SEQ ID NO: 11, respectively). In some embodiments, the anti-PD-L1 antibody comprises a heavy chain variable domain comprising the amino acid sequence of SEQ ID NO: 8 and a light chain variable domain comprising the amino acid sequence of SEQ ID NO: 9). In some embodiments, the anti-PD-L1 antibody comprises a heavy chain comprising the amino acid sequence of SEQ ID NO: 10 and a light chain comprising the amino acid sequence of SEQ ID NO: 11.

In some embodiments, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO2011/066389 and US2013/034559. Durvalumab comprises a heavy chain and a light chain sequence, wherein:

(a) the heavy chain comprises the amino acid
sequence:
(SEQ ID NO: 14)
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPEFEGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPASIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPG,
and
(b) the light chain comprises the amino acid
sequence:
(SEQ ID NO: 15)
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In some embodiments, the anti-PD-L1 antibody comprises the six CDR sequences from SEQ ID NO: 14 and SEQ ID NO: 15 (e.g., the three heavy chain CDRs from SEQ ID NO: 14 and the three light chain CDRs from SEQ ID NO: 15). In some embodiments, the anti-PD-L1 antibody comprises the heavy chain variable domain from SEQ ID NO: 14 and the light chain variable domain from SEQ ID NO: 15.

In some embodiments, the anti-PD-L1 antibody is MDX-1105 (Bristol Myers Squibb). MDX-1105, also known as BMS-936559, is an anti-PD-L1 antibody described in WIPO patent publication number WO2007/005874.

In some embodiments, the anti-PD-L1 antibody is LY3300054 (Eli Lilly).

In some embodiments, the anti-PD-L1 antibody is STI-A1014 (Sorrento). STI-A1014 is a human anti-PD-L1 antibody.

In some embodiments, the anti-PD-L1 antibody is KN035 (Suzhou Alphamab). KN035 is single-domain antibody (dAB) generated from a camel phage display library.

In some embodiments, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PD-L1 antibody is CX-072 (CytomX Therapeutics).

In some embodiments, the anti-PD-L1 antibody comprises the six CDR sequences (e.g., the three heavy chain CDRs and the three light chain CDRs) and/or the heavy chain variable domain and light chain variable domain from a PD-L1 antibody described in US Patent Publication Number 20160108123 (Assigned to Novartis); WIPO Patent Publication Numbers WO2016/000619 (Applicant: Beigene), WO2012/145493 (Applicant: Amplimmune), WO2013/181634 (Applicant: Sorrento), and WO2016/061142 (Applicant: Novartis), and U.S. Pat. No. 9,205,148 (Assigned to MedImmune).

Formulations

Each of the oncolytic virus and anti-PD-L1 antibody used in the methods of the present disclosure can be formulated into a composition suitable for administration to the subject. In exemplary aspects, each of the oncolytic virus and anti-PD-L1 antibody can be formulated with one or more agents which enhance the chemico-physico features of the oncolytic virus and/or anti-PD-L1 antibody, e.g., via stabilizing the the oncolytic virus and/or anti-PD-L1 antibody at certain temperatures, e.g., room temperature, increasing shelf life, reducing degradation, e.g., oxidation protease mediated degradation, increasing half-life of the the oncolytic virus and/or anti-PD-L1 antibody, etc. In exemplary aspects of the present disclosure, the the oncolytic virus and/or anti-PD-L1 antibody may be formulated into a composition additionally comprising a pharmaceutically acceptable carrier, diluents, or excipient. In some embodiments, the oncolytic virus and/or anti-PD-L1 antibody is formulated into a pharmaceutical composition comprising the oncolytic virus and/or anti-PD-L1 antibody, along with a pharmaceutically acceptable carrier, diluent, or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans. The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as bcnzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In exemplary embodiments, the pH of the pharmaceutical composition is between 5.5 and 7.5.

In exemplary aspects, the oncolytic virus is talimogene laherparepvec and is formulated with disodium hydrogen phosphate dihydrate, sodium dihydrogen phospohate dihydrate, sodium chloride, myo-inositol, sorbitol, and water for injection. In exemplary aspects, the composition comprises 10⁶ PFU or 10⁸ PFU per mL talimogene laherparepvec, 15.4 mg per mL disodium hydrogen phosphate dihydrate, 2.44 mg per mL sodium dihydrogen phospohate dihydrate, 8.5 mg per mL sodium chloride, 40 mg per mL myo-inositol, 20 mg per mL sorbitol, and water for injection.

In exemplary aspects, the anti-PD-L1 antibody is atezolizumab and is formulated with glacial acetic acid, L-histidine, sucrose, and polysorbate 20. In exemplary aspects, the composition comprises 60 mg per mL atezolizumab, 16.5 mg per mL glacial acetic acid, 62 mg per mL L-histidine, 821.6 mg per mL sucrose, and 8 mg per mL polysorbate 20. In exemplary asepcts, the composition of atezolizumab has a pH of 5.8.

Routes of Administration

With regard to the methods of the present disclosure, each of the oncolytic virus and anti-PD-L1 antibody can be administered to the subject via any suitable route of administration. For example, each of the oncolytic virus and anti-PD-L1 antibody can be administered to a subject via parenteral, nasal, oral, pulmonary, topical, vaginal, or rectal administration. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl -imidazoline quaternary ammonium salts, and (e) mixtures thereof.

The parenteral formulations in some embodiments contain from about 0.5% to about 25% by weight of the active agent of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

In exemplary aspects, the oncolytic virus is administered by injection into cutaneous, subcutaneous, and/or nodal lesions. In exemplary aspects, the oncolytic virus is talimogene laherparepvec and is administered by intralesional injection to liver metastases or cutaneous, subcutaneous and nodal tumor lesions, or both. In exemplary aspects, the oncolytic virus is not administered via intravenous administration. In exemplary aspects, the oncolytic virus is administered intrahepatically, e.g., via intrahepatic injection (e.g., directly into the liver). In exemplary aspects, the oncolytic virus is administered to one or more injectable liver lesions in the subject. In exemplary aspects, the oncolytic virus is administered by imaged guided injection (e.g., ultrasound or computerized tomography (CT)) into injectable liver lesions. In exemplary aspects, the oncolytic virus is administered intratumorally, e.g., administered by imaged guided injection (e.g., ultrasound or CT) into a tumor. In exemplary aspects, the liver lesion is non-resectable.

In exemplary aspects, the anti-PD-L1 antibody is administered to the subject intravenously, e.g., via intravenous infusion. In exemplary aspects, the anti-PD-L1 antibody is administered to the subject via intravenous infusion over about 15 minutes to about 2 hours. In exemplary aspects, the anti-PD-L1 antibody is administered to the subject via intravenous infusion over about 30 minutes to about 100 minutes. In exemplary aspects, the anti-PD-L1 antibody is administered to the subject via intravenous infusion over about 45 minutes to about 75 minutes. In exemplary aspects, the anti-PD-L1 antibody is administered to the subject via intravenous infusion over about 60 minutes. In exemplary aspects, the methods of the present disclosure comprise administering at least one addition dose of the anti-PD-L1 antibody, optionally, via intravenously over a shorter infusion time. In exemplary aspects, a first dose of the anti-PD-L1 antibody is given to the subject via intravenous infusion over about 45 minutes to about 75 minutes (e.g., about 60 minutes) and one or more subsequent administrations to the subject are given to the subject via intravenous infusion over about 20 minutes to about 40 minutes (e.g., about 30 minutes). In exemplary aspects, the one or more subsequent administrations are given to the subject about 21-24 days following the first dose. In exemplary aspects, each dose given to the subject is about 1000 mg to about 1500 mg or about 1150 mg to about 1350 mg, e.g., about 1200 mg.

Dosages

For purposes of the present disclosure, the amount or dose of each of the oncolytic virus and anti-PD-L1 antibody administered to the subject should be sufficient to effect, e.g., a therapeutic or prophylactic response, in the subject or animal over a reasonable time frame. For example, the dose of each of the oncolytic virus and anti-PD-L1 antibody should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular oncolytic virus and anti-PD-L1 antibody and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of each of the oncolytic virus and anti-PD-L1 antibody to a mammal among a set of mammals, each set of which is given a different dose of the active agent, could be used to determine a starting dose to be administered to a mammal The extent to which cancer is treated upon administration of a certain dose can be represented by, for example, the cytotoxicity of the active agent or the extent of tumor regression achieved with the oncolytic virus and anti-PD-L1 antibody in a mouse xenograft model. Methods of measuring cytotoxicity and methods of assaying tumor regression are known in the art. See, the examples set forth herein.

The dose will be determined by the existence, nature and extent of any adverse side effects that might accompany the administration of a particular oncolytic virus and anti-PD-L1 antibody. Typically, the attending physician will decide the dosage with which to treat each individual patient, taking into consideration a variety of factors, such as age, body weight, general health, diet, sex, therapeutic agents to be administered, route of administration, and the severity of the condition being treated.

By way of example and not intending to limit the present disclosure, the dose of the oncolytic virus is about 10² PFU/mL to about 10¹² PFU/mL. In exemplary aspects, the dose of the oncolytic virus is about 10⁴ PFU/mL to about 10¹⁰ PFU/mL. In exemplary aspects, the dose of the oncolytic virus is about 10⁶ PFU/mL to about 10⁸ PFU/mL. In exemplary aspects, the first dose of the oncolytic virus is about 10⁶ PFU/mL or about 10⁷ PFU/mL. In exemplary aspects, subsequent doses of the oncolytic virus are about 10⁸ PFU/mL.

In exemplary aspects, the method comprises administering to the subject an initial dose of the oncolytic virus followed by a second dose. In exemplary instances, the initial dose is lower than the second dose. In exemplary aspects, the initial dose is no more than about half the second dose. In exemplary aspects, the initial dose is no more than a quarter of the second dose. In exemplary aspects, the initial dose is no more than 1/10^{th} the second dose. In exemplary aspects, the initial dose is no more than 1/100^{th} the second dose. In exemplary aspects, the method comprises administering to the subject at least one additional dose following the second dose, and, optionally, each additional dose is approximately the same amount as the second dose. In exemplary aspects, the method comprises administering to the subject two, three, or four additional doses following the second dose. In exemplary aspects, each dose of the oncolytic virus given to the subject is given approximately once every 21-24 days. In exemplary aspects, the initial dose of the oncolytic virus is not more than 4.0 ml of a solution comprising the oncolytic virus at a concentration of about 10⁶ PFU/ml. In other aspects, the initial dose of the oncolytic virus is not more than 8.0 ml of a solution comprising the oncolytic virus at a concentration of about 10⁶ PFU/ml. In exemplary aspects, the initial dose of the oncolytic virus is 0.5- 8.0 ml (e.g., 0.5, 1.0, 1.5, 2.0, 2.5, 3.0, 3.5, 4.0, 4.5, 5.0, 5.5, 6.0, 6.5, 7.0, 7.5, or 8.0 mL) of a solution comprising the oncolytic virus at a concentration of about 10⁶ PFU/ml. In exemplary aspects, the second dose of the oncolytic virus is not more than 4.0 ml of a solution comprising the oncolytic virus at a concentration of about 10⁸ PFU/ml. In exemplary aspects, the second dose of the oncolytic virus is not more than 8.0 ml of a solution comprising the oncolytic virus at a concentration of about 10⁸ PFU/ml. In exemplary aspects, the second dose of the oncolytic virus is 0.5- 8.0 ml (e.g., 0.5, 1.0, 1.5, 2.0, 2.5, 3.0, 3.5, 4.0, 4.5, 5.0, 5.5, 6.0, 6.5, 7.0, 7.5, or 8.0 mL) of a solution comprising the oncolytic virus at a concentration of about 10⁸ PFU/ml. In exemplary aspects, each additional dose of the oncolytic virus subsequently administered to the second dose is not more than 4.0 ml of a solution comprising the oncolytic virus at a concentration of about 10⁸ PFU/ml. In exemplary aspects, each additional dose of the oncolytic virus subsequently administered to the second dose is not more than 8.0 ml of a solution comprising the oncolytic virus at a concentration of about 10⁸ PFU/ml. In exemplary aspects, the additional dose of the oncolytic virus is 0.5- 8.0 ml (e.g., 0.5, 1.0, 1.5, 2.0, 2.5, 3.0, 3.5, 4.0, 4.5, 5.0, 5.5, 6.0, 6.5, 7.0, 7.5, or 8.0 mL) of a solution comprising the oncolytic virus at a concentration of about 10⁸ PFU/ml. In exemplary aspects, the administered volume of the oncolytic virus is determined based on the tumor lesions of the subject. See the teachings below under “Regimen”.

By way of example and not intending to limit the present disclosure, the dose of the anti-PD-L1 antibody is between about 500 mg to about 5000 mg. In exemplary aspects, the dose of the anti-PD-L1 antibody is between about 800 mg to about 2500 mg. In exemplary aspects, the does of the anti-PD-L1 antibody is about 1000 mg to about 1400 mg, e.g., about 1200 mg. In exemplary aspects, the dose of the anti-PD-L1 antibody is about 1 mg/kg to about 20 mg/kg. In exemplary aspects, the dose of the anti-PD-L1 antibody is about 10 mg/kg to about 20 mg/kg. In exemplary aspects, the dose of the anti-PD-L1 antibody is about 12.5 mg/kg to about 17.5 mg/kg. In exemplary aspects, the dose of the anti-PD-L1 antibody is about 15 mg/kg. In exemplary aspects, the methods of the present disclosure comprise administering to the subject more than one dose of the anti-PD-L1 antibody. In exemplary aspects, each dose of the anti-PD-L1 antibody administered to the subject (e.g., approximately given once every 21-24 days) is approximately the same.

Regimen

In exemplary aspects, the oncolytic virus is administered simulataneously as the anti-PD-L1 antibody. In exemplary aspects, the oncolytic virus is administered separately from the anti-PD-L1 antibody. For example, the oncolytic virus is administered before the anti-PD-L1 antibody or the oncolytic virus is administered after the anti-PD-L1 antibody. In exemplary aspects, the oncolytic virus is administered by imaged guided injection. In exemplary aspects, the anti-PD-L1 antibody is administered intravenously.

In exemplary aspects, the oncolytic virus is administered more than once. In exemplary aspects, the oncolytic virus is administered once a week or once every 2, 3, or 4 weeks. In exemplary aspects, the oncolytic virus is administered once every 18, 19, 20, 21, 22, 23, or 24 days. In exemplary instances, the oncolytic virus is administered once every 21 (+3) days or once every 21 (±3) days. In exemplary instances, the oncolytic virus is administered once every 18-2ldays. In exemplary instances, the oncolytic virus is administered once every 21-24 days. In exemplary instances, the oncolytic virus is administered for 1-6 cycles and the first cycle ends at 21(±3) days following the first administration, the 2 cycle accordingly begins at the beginning of Week 4 (+3 days) and the 2^{nd} administration occurs at the beginning of Week 4 (+3 days). In exemplary instances, the 2^{nd} cycle ends at 21 (±2) days and any subsequent administration occurs every 21 (±3) days. In exemplary instances, the oncolytic virus is talimogene laherparepvec which is administered once every 18-21 days or once every 21-24 days, e.g., (once every 18, 19, 20, 21, 22, 23, or 24 days). In exemplary instances, the oncolytic virus (e.g., talimogene laherparepvec) is administered once every 21-24 days and is given to the subject a total of 2, 3, 4, 5, or 6 times. In exemplary aspects, the oncolytic virus (e.g., talimogene laherparepvec) is administered more than 6 times, e.g., 7, 8, 9, 10, 11, or 12 times. In exemplary aspects, the oncolytic virus (e.g., talimogene laherparepvec) is administered for 6 months or 1 year, if not longer. In exemplary instances, talimogene laherparepvec is administered for 1-6 cycles and the first cycle ends at 21(±3) days following the first administration, the 2^{nd} cycle accordingly begins at the beginning of Week 4 (+3 days) and the 2^{nd} administration occurs at the beginning of Week 4 (+3 days). In exemplary instances, the 2^{nd} cycle ends at 21 (±2) days and any subsequent administration occurs every 21 (±3) days.

In exemplary instances, the oncolytic virus is administered to the subject at an initial dose followed by a second dose, optionally, wherein the initial dose is lower than the second dose. In certain aspects, the oncolytic virus is administered intratumorally. In exemplary aspects, the second dose is administered about 14 days or more after the initial dose. In some aspsects, the second dose is administered to the subject about 21 days or more after the initial dose. In exemplary instances, the second dose is administered to the subject about 21, about 22, about 23, about 24, about 25, or about 26 days, or more, after the initial dose. In some aspects, the second dose is administered to the subject about 27 days to about 31 days after the initial dose. In some aspects, at least one subsequent dose (optionally, 2, 3, 4, or more subsequent doses) of the oncolytic virus is administered after the second dose. In certain instances, the method comprises administering the subsequent doses about every 21 days after administration of the second dose. In exemplary aspects, the initial dose of the oncolytic virus is not more than 4.0 ml of a solution comprising the oncolytic virus at a concentration of about 10⁶ PFU/ml. In exemplary aspects, the second dose of the oncolytic virus is not more than 4.0 ml of a solution comprising the oncolytic virus at a concentration of about 10⁶ PFU/ml. In exemplary aspects, the one or more subsequent doses of the oncolytic virus is/are not more than 4.0 ml of a solution comprising the oncolytic virus at a concentration of about 10⁶ PFU/ml.

In exemplary embodiments, the oncolytic virus (e.g., talimogene laherparepvec) is administered by imaged guided injection (either ultrasound or CT) into injectable liver lesions. The first cycle of the oncolytic virus (e.g., talimogene laherparepvec) in exemplary instances is 21 (±3) days, e.g., 18, 19, 20, 21, 22, 23, 24 days. In some aspects, subsequent cycles of the oncolytic virus (e.g., talimogene laherparepvec) is 21(+/- 3 days), e.g., 18, 19, 20, 21, 22, 23, 24 days. For example, on cycle 1, day 1, the first dose of the oncolytic virus (e g , talimogene laherparepvec) is up to 4.0 mL of 10⁶ PFU/mL, and, during the second cycle, the oncolytic virus (e.g., talimogene laherparepvec) is administered up to 4.0 mL of 10⁸ PFU/mL at week 4 of the study (±3 days). During subsequent cycles, the oncolytic virus (e.g., talimogene laherparepvec) is administered up to 4.0 mL of 10⁸ PFU/mL every 21 days (±3 days) thereafter.

In exemplary aspects, the administered volume of the oncolytic virus is determined based on the tumor lesions of the subject. In exemplary aspects, the maximum volume of talimogene laherparepvec to be administered at any treatment visit is 4.0 mL for any individual tumor lesion or for all tumor lesions combined. The volume of the oncolytic virus (e.g., talimogene laherparepvec) to be injected into the tumor(s) can depend on the longest diameters of the tumor(s) and necrotic core of the tumor(s) (if applicable) and should be dosed according to the injection volume guideline in Table 1.

TABLE 1

| Individual Tumor Diameter^{a} | | | | |
|---|---|---|---|---|
| <75% tumor diameter | | ≥75% tumor diameter | | Maximum Injection |
| necrotic^{b} | | necrotic^{b} | | Volume |
| ≥4 | cm | ≥5 | cm | 4 mL |
| ≥2 to <4 | cm | ≥2.5 to <5 | cm | 2 mL |
| <1.5 | cm | <2.5 | cm | 1 mL |

^{a}Longest tumor diameter assessed by ultrasound or CT in preparation for injection guidance.
^{b}Based on longest necrotic core diameter divided by longest tumor diameter from most recent multiphase computerized tomography (CT) or magnetic resonance imaging (MRI)

In exemplary aspects, the volume of the oncolytic virus (e g , talimogene laherparepvec) to be injected into the tumor(s) is based on the longest diameters of cutaneous, subcutaneous and nodal tumor lesions assessed on the day of treatment according to Table 2:

TABLE 2

| Tumor Size (longest dimension) | Maximum Injection Volume |
|---|---|
| >5.0 cm | 4.0 mL |
| >2.5 cm to 5.0 cm | 2.0 mL |
| >1.5 cm to 2.5 cm | 1.0 mL |
| 0.5 cm to 1.5 cm | 0.5 mL |

In exemplary aspects, talimogene laherparepvec is administered by imaged guided injection (either ultrasound or CT) into injectable liver lesions for at least two or at least three cycles (e.g., 1-6 cycles or more). The first cycle of talimogene laherparepvec in some aspect is 21 (+3) days. Subsequent cycles of talimogene laherparepvec in some aspects is 21(+/−3 days). In exemplary instances, on cycle 1, day 1, the first dose of talimogene laherparepvec is up to 4.0 mL of 10⁶ PFU/mL, and during the second cycle, talimogene laherparepvec is administered up to 4.0 mL of 10⁸ PFU/mL at week 4 of the study (+3 days). During subsequent cycles, talimogene laherparepvec in some aspects is administered up to 4.0 mL of 10⁸ PFU/mL every 21 days (±3 days) thereafter

In exemplary aspects, the anti-PD-L1 antibody is administered more than once. In exemplary aspects, anti-PD-L1 antibody is administered once a week or once every 2, 3, or 4 weeks. In exemplary aspects, the anti-PD-L1 antibody is administered once every 18, 19, 20, 21, 22, 23, or 24 days. In exemplary instances, the anti-PD-L1 antibody is administered once every 18-24 days. In exemplary instances, the anti-PD-L1 antibody is administered once every 21-24 days. In exemplary instances, the anti-PD-L1 antibody is atezolizumab which is administered once every 21-24 days. In exemplary instances, the anti-PD-L1 antibody (e.g., atezolizumab) is administered once every 21-24 days and is given to the subject a total of 2, 3, 4, 5, or 6 times. In exemplary aspects, the anti-PD-L1 antibody (e.g., atezolizumab) is administered more than 6 times, e.g., 7, 8, 9, 10, 11, or 12 times. In exemplary aspects, the anti-PD-L1 antibody (e.g., atezolizumab) is administered for 6 months or 1 year, if not longer. In exemplary instances, the anti-PD-L1 antibody (e.g., atezolizumab) is administered for more than one cycle and the first cycle ends at 21(±3) days following the first administration, and a subsequent administration occurs at 21 (±3) days.

In exemplary embodiments, the anti-PD-L1 antibody (e.g., atezolizumab) is administered about every 21 (±3) days. For example, the first cycle of anti-PD-L1 antibody (e.g., atezolizumab) is 21 (±3) days and subsequent cycles of anti-PD-L1 antibody (e.g., atezolizumab) is 21 (±3) days. The dose level of anti-PD-L1 antibody (e.g., atezolizumab) in exemplary aspects is 1200 mg administered by, for example, intravenous infusionln exemplary aspects, the initial dose of atezolizumab (day 1, cycle 1) is delivered over 60 (±15) minutes. In exemplary aspects, if the first dose is tolerated without infusion-associated adverse events, the second dose (cycle 2) is delivered over 30 (±10) minutes. If the 30-minute intravenous infusion is well tolerated, all subsequent doses can be delivered over 30 (±10) minutes.

In exemplary aspects, the first cycle of atezolizumab will be 21 (+3) days, e.g., 18, 19, 20, 21, 22, 23, or 24 days. Subsequent cycles of atezolizumab in some instances is 21 (±3) days, e.g., 18, 19, 20, 21, 22, 23, or 24 days.. In exemplary aspects, the dose level of atezolizumab is 1200 mg administered by intravenous infusion. In exemplary aspects, the initial dose of atezolizumab (day 1, cycle 1) is delivered over 60 (±15) minutes. If the first dose is tolerated without infusion-associated adverse events, the second dose (cycle 2) may be delivered over 30 (±10) minutes. If the 30-minute intravenous infusion is well tolerated, all subsequent doses may be delivered over 30 (±10) minutes. The subjects' vital signs (heart rate, respiratory rate, blood pressure, and temperature) should be determined up to 60 minutes before each atezolizumab intravenous infusion. Vital signs should also be obtained during or after the atezolizumab intravenous infusion if clinically indicated.

In exemplary instances, the method comprises intravenously administering the PD-L1 antibody to the subject. In some aspects, the method comprises administering the PD-L1 antibody to the subject over about 45 minutes to about 75 minutes (e.g., about 45 minutes, about 50 minutes, about 55 minutes, about 60 minutes, about 65 minutes, about 70 minutes, about 75 minutes, about 45 minutes to about 70 minutes, about 45 minutes to about 65 minutes, about 45 minutes to about 60 minutes, about 45 minutes to about 55 minutes, about 45 minutes to about 50, about 50 minutes to tabout 75 minutes, about 55 minutes to about 75 minutes about 60 minutes to about 75 minutes, about 65 minutes to about 75 minutes, about 70 minutes to about 75 minutes). In certain instances, the method further comprises administering a second administration of the PD-L1 antibody. In some exemplary aspects, the second administration occurs over about 20 minutes to about 40 minutes (e.g., about 20 minutes to about 35 minutes, about 20 minutes to about 30 minutes about 20 minutes to about 25 minutes, about 25 minutes to about 40 minutes, about 30 minutes to about 40 minutes, about 35 minutes to about 40 minutes. In certain aspects, the second administration of the PD-L1 antibody occurs about 21 days to about 24 days after the first administration, e.g., about 21 days, about 22 days, about 23 days, about 24 days after the first administration. In certain instances, the second administration of the PD-L1 antibody occurs about 21 days after the first administration. In exemplary aspects, at least one subsequent administration of the PD-L1 antibody is given to the subject after the second administration. In some aspects, at least 2, at least 3, at least 4 or more subsequent administrations of the PD-L1 antibody are given to the subject after the second administration. The subsequent administration(s) of the PD-L1 antibody occur(s) about 18 days to about 24 days after the second administration in some instances. Optionally, the PD-L1 antibody is administered at a dose of about 1000 mg to about 1500 mg (e.g., about 1000 mg to about 1450 mg, about 100 mg to about 1400 mg, about 1000 mg to about 1350 mg, about 1000 mg to about 1300 mg, about 1000 mg to about 1250 mg, about 1000 mg to about 1200 mg, about 1000 mg to about 1150 mg, about 1000 mg to about 1100 mg, about 1000 mg to about 1050 mg, about 1050 mg to about 1500 mg, about 1100 mg to about 1500 mg, about 1150 mg to about 1500 mg, about 1200 mg to about 1500 mg, about 1250 mg to about 1500 mg, about 1300 mg to about 1500 mg, about 1350 mg to about 1500 mg, about 1400 mg to about 1500 mg, about 1450 mg to about 1500 mg. In some aspects, the PD-L1 antibody is administered at a dose of about 1150 mg to about 1350 mg, optionally, about 1200 mg.

Additional Components

In some embodiments, the method comprises administering another therapeutic agent. The therapeutic agent may be any of those known in the art. Examples of therapeutic agents that are contemplated herein include, but are not limited to, natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT₄ partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A₂ inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopoptides, lincos amides, macrolies, oxazolidinones, penicillins, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamind reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opoid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The therapeutic agent can be a cytokine, lymphokine, growth factor, or other hematopoietic factor, including, but not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

In some embodiments, the therapeutic agent is a cytotoxic agent. The cytotoxic agent is any molecule (chemical or biochemical) which is toxic to a cell. In some aspects, when a cytotoxic agent is administered, the results obtained are synergistic. That is to say, the effectiveness of the combination therapy of the oncolytic virus, anti-PD-L1 antibody, and the cytotoxic agent is synergistic, i.e., the effectiveness is greater than the effectiveness expected from the additive individual effects of each. Therefore, the dosage of the cytotoxic agent can be reduced and thus, the risk of the toxicity problems and other side effects is concomitantly reduced. In some embodiments, the cytotoxic agent is a chemotherapeutic agent. Chemotherapeutic agents are known in the art and include, but not limited to, platinum coordination compounds, topoisomerase inhibitors, antibiotics, antimitotic alkaloids and difluoronucleosides, as described in U.S. Pat. No. 6,630,124.

In some embodiments, the chemotherapeutic agent is a platinum coordination compound. The term “platinum coordination compound” refers to any tumor cell growth inhibiting platinum coordination compound that provides the platinum in the form of an ion. In some embodiments, the platinum coordination compound is cis-diamminediaquoplatinum (II)-ion; chloro(diethylenetriamine)-platinum(II)chloride; dichloro(ethylenediamine)-platinum(II), diammine(1,1-cyclobutanedicarboxylato) platinum(II) (carboplatin); spiroplatin; iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-caroxyphthalato)(1,2-diaminocyclohexane)platinum(II); (1,2-diaminocyclohexane)-(isocitrato)platinum(II); (1,2-diaminocyclohexane)cis(pyruvato)platinum(II); (1,2-diaminocyclohexane)oxalatoplatinum(II); ormaplatin; or tetraplatin.

In some embodiments, cisplatin is the platinum coordination compound employed in the compositions and methods of the present present disclosure. Cisplatin is commercially available under the name PLATINOL™ from Bristol Myers-Squibb Corporation and is available as a powder for constitution with water, sterile saline or other suitable vehicle. Other platinum coordination compounds suitable for use in the present present disclosure are known and are available commercially and/or can be prepared by conventional techniques. Cisplatin, or cis-dichlorodiammineplatinum II, has been used successfully for many years as a chemotherapeutic agent in the treatment of various human solid malignant tumors. More recently, other diamino-platinum complexes have also shown efficacy as chemotherapeutic agents in the treatment of various human solid malignant tumors. Such diamino-platinum complexes include, but are not limited to, spiroplatinum and carboplatinum. Although cisplatin and other diamino-platinum complexes have been widely used as chemotherapeutic agents in humans, they have had to be delivered at high dosage levels that can lead to toxicity problems such as kidney damage.

In some embodiments, the chemotherapeutic agent is a topoisomerase inhibitor. Topoisomerases are enzymes that are capable of altering DNA topology in eukaryotic cells. They are critical for cellular functions and cell proliferation. Generally, there are two classes of topoisomerases in eukaryotic cells, type I and type II. Topoisomerase I is a monomeric enzyme having a molecule weight of approximately 100,000 kDa. The enzyme binds to DNA and introduces a transient single-strand break, unwinds the double helix (or allows it to unwind), and subsequently reseals the break before dissociating from the DNA strand. Various topoisomerase inhibitors have recently shown clinical efficacy in the treatment of humans afflicted with ovarian, cancer, esophageal cancer or non-small cell lung carcinoma.

In some aspects, the topoisomerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin exhibits tumor cell growth inhibiting activity against a number of tumor cells. Compounds of the camptothecin analog class are typically specific inhibitors of DNA topoisomerase I. By the term “inhibitor of topoisomerase” is meant any tumor cell growth inhibiting compound that is structurally related to camptothecin. Compounds of the camptothecin analog class include, but are not limited to; topotecan, irinotecan and 9-amino-camptothecin.

In additional embodiments, the cytotoxic agent is any tumor cell growth inhibiting camptothecin analog claimed or described in: U.S. Pat. No. 5,004,758, issued on Apr. 2, 1991 and European Patent Application Number 88311366.4, published on Jun. 21, 1989 as Publication Number EP 0 321 122; U.S. Pat. No. 4,604,463, issued on Aug. 5, 1986 and European Patent Application Publication Number EP 0 137 145, published on Apr. 17, 1985; U.S. Pat. No. 4,473,692, issued on Sep. 25, 1984 and European Patent Application Publication Number EP 0 074 256, published on Mar. 16, 1983; U.S. Pat. No. 4,545,880, issued on Oct. 8, 1985 and European Patent Application Publication Number EP 0 074 256, published on Mar. 16, 1983; European Patent Application Publication Number EP 0 088 642, published on Sep. 14, 1983; Wani et al., J. Med. Chem., 29, 2358-2363 (1986); Nitta et al., Proc. 14th International Congr. Chemotherapy, Kyoto, 1985, Tokyo Press, Anticancer Section 1, p. 28-30, especially a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776, issued on Aug. 3, 1982; U.S. Patent Application Ser. No. 581,916, filed on Sep. 13, 1990 and European Patent Application Publication Number EP 418 099, published on Mar. 20, 1991; U.S. Pat. No. 4,513,138, issued on Apr. 23, 1985 and European Patent Application Publication Number EP 0 074 770, published on Mar. 23, 1983; U.S. Pat. No. 4,399,276, issued on Aug. 16, 1983 and European Patent Application Publication Number 0 056 692, published on Jul. 28, 1982; the entire disclosure of each of which is hereby incorporated by reference. All of the above-listed compounds of the camptothecin analog class are available commercially and/or can be prepared by conventional techniques including those described in the above-listed references. The topoisomerase inhibitor may be selected from the group consisting of topotecan, irinotecan and 9-aminocamptothecin.

The preparation of numerous compounds of the camptothecin analog class (including pharmaceutically acceptable salts, hydrates and solvates thereof) as well as the preparation of oral and parenteral pharmaceutical compositions comprising such a compounds of the camptothecin analog class and an inert, pharmaceutically acceptable carrier or diluent, is extensively described in U.S. Pat. No. 5,004,758, issued on Apr. 2, 1991 and European Patent Application Number 88311366.4, published on Jun. 21, 1989 as Publication Number EP 0 321 122, the teachings of which are incorporated herein by reference.

In still yet other embodiments of the present disclosure, the chemotherapeutic agent is an antibiotic compound. Suitable antibiotic include, but are not limited to, doxorubicin, mitomycin, bleomycin, daunorubicin and streptozocin.

In some embodiments, the chemotherapeutic agent is an antimitotic alkaloid. In general, antimitotic alkaloids can be extracted from Cantharanthus roseus, and have been shown to be efficacious as anticancer chemotherapy agents. A great number of semi-synthetic derivatives have been studied both chemically and pharmacologically (see, O. Van Tellingen et al, Anticancer Research, 12, 1699-1716 (1992)). The antimitotic alkaloids of the present present disclosure include, but are not limited to, vinblastine, vincristine, vindesine, Taxol and vinorelbine. The latter two antimitotic alkaloids are commercially available from Eli Lilly and Company, and Pierre Fabre Laboratories, respectively (see, U.S. Pat. No. 5,620,985). In a preferred aspect of the present present disclosure, the antimitotic alkaloid is vinorelbine.

In other embodiments of the present disclosure, the chemotherapeutic agent is a difluoronucleoside. 2′-deoxy-2′,2′-difluoronucleosides are known in the art as having antiviral activity. Such compounds are disclosed and taught in U.S. Pat. Nos. 4,526,988 and 4,808614. European Patent Application Publication 184,365 discloses that these same difluoronucleosides have oncolytic activity. In certain specific aspects, the 2′-deoxy-2′,2′-difluoronucleoside used in the compositions and methods of the present present disclosure is 2′-deoxy-2′,2′-difluorocytidine hydrochloride, also known as gemcitabine hydrochloride. Gemcitabine is commercially available or can be synthesized in a multi-step process as disclosed and taught in U.S. Pat. Nos. 4,526,988; 4,808,614; and 5,223,608, the teachings of which are incorporated herein by reference.

Use

The methods of the present disclosure provide treatment to the indicated subject(s). As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating triple negative cancer or colorectal cancer of the present disclosure can provide any amount or any level of treatment. Furthermore, the treatment provided by the method of the present disclosure may include treatment of one or more conditions or symptoms or signs of the cancer being treated. Also, the treatment provided by the methods of the present disclosure may encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods treat by way of delaying the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods treat by way increasing the survival of the subject.

In exemplary aspects, the methods of the present disclosures decrease tumor burden in a subject with triple negative breast cancer or colorectal cancer. As used herein, the term “tumor burden” refers to the sum of diameter of target lesions +sum of diameter of up to 10 (maximum 5 per organ) new, measurable lesions. In exemplary aspects, “tumor burden” refers to the sum of diameters of target lesions identified at baseline plus the sum of diameters of up to 10 (maximum 5 per organ) new measurable lesions (for which the longest diameter is >10 mm for non-nodal lesions or the short axis is >15 mm for non-nodal lesions). In exemplary aspects, the tumor burden is decreased by at least or about a 10% (e.g., at least or about a 20%, at least or about a 30%, at least or about a 40%, at least or about a 50%, at least or about a 60%, at least or about a 70%, at least or about a 80%, at least or about a 90%, at least or about a 95%, at least or about a 98%).

In some aspects, the methods of the present disclosures result in progression free survival. In exemplary instances, the methods of the present disclosures result in progression free survival for at least or about 1 month, at least or about 2 months, at least or about 3 months, at least or about 4 months, at least or about 5 months, at least or about 6 months, at least or about 7 months, at least or about 8 months, at least or about 9 months, at least or about 10 months, at least or about 11 months, at least or about 12 months or more (e.g., at least or about 13 months, at least or about 13 months, at least or about 13 months, at least or about 13 months, at least or about 14 months, at least or about 15 months, at least or about 16 months, at least or about 17 months, at least or about 18 months, at least or about 19 months, at least or about 20 months, at least or about 21 months, at least or about 22 months, at least or about 23 months, at least or about 24 months). Optionally, the progression free survival is even greater than about 24 months, e.g., greater than about 30 months, greater than about 36 months, greater than about 48 months, greater than about 60 months.

In exemplary instances, the methods of the present disclosures result in an increase in overall survival. In some instances, the the present disclosures result in an increase in overall survival of at least or about 1 month, at least or about 2 months, at least or about 3 months, at least or about 4 months, at least or about 5 months, at least or about 6 months, at least or about 7 months, at least or about 8 months, at least or about 9 months, at least or about 10 months, at least or about 11 months, at least or about 12 months or more (e.g., at least or about 13 months, at least or about 13 months, at least or about 13 months, at least or about 13 months, at least or about 14 months, at least or about 15 months, at least or about 16 months, at least or about 17 months, at least or about 18 months, at least or about 19 months, at least or about 20 months, at least or about 21 months, at least or about 22 months, at least or about 23 months, at least or about 24 months). Optionally, the overall survival is even greater than about 24 months, e.g., greater than about 30 months, greater than about 36 months, greater than about 48 months, greater than about 60 months.

In exemplary instances, the methods of the present disclosures result in both progression free survival and an increase in overall survival. In some aspects, one or both is at least or about 1 month, at least or about 2 months, at least or about 3 months, at least or about 4 months, at least or about 5 months, at least or about 6 months, at least or about 7 months, at least or about 8 months, at least or about 9 months, at least or about 10 months, at least or about 11 months, at least or about 12 months or more (e.g., at least or about 13 months, at least or about 13 months, at least or about 13 months, at least or about 13 months, at least or about 14 months, at least or about 15 months, at least or about 16 months, at least or about 17 months, at least or about 18 months, at least or about 19 months, at least or about 20 months, at least or about 21 months, at least or about 22 months, at least or about 23 months, at least or about 24 months). Optionally, one or both of the overall survival or the progression free survival is even greater than about 24 months, e.g., greater than about 30 months, greater than about 36 months, greater than about 48 months, greater than about 60 months.

Subjects

In some embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

In exemplary aspects, the human is a male or female 18 years old or older. In exemplary aspects, the subject has a confirmed diagnosis of triple negative breast cancer or colorectal cancer. In exemplary instances, the subject has a confirmed diagnosis of triple negative breast cancer with liver metastases or colorectal cancer with liver metastases. In exemplary aspects, the human subject is a female with invasive breast cancer. In exemplary aspects, the subject has metastases in the lung, brain, liver, and/or bone. In exemplary aspects, the subject has a BRCA1 mutation.

In exemplary aspects, the subject has colorectal cancer optionally, Stage I, II, III, or IV, optionally with synchronous liver metastases at initial diagnosis or metachronous liver metastases. In exemplary aspects, the liver is the only metastatic site. In exemplary aspects, the colorectal cancer is a microsatellite instable colorectal cancer or a sporadic colorectal cancer. In exemplary aspects, the colorectal cancer is a microsatellite stable colorectal cancer or a familial colorectal cancer.

In some aspects, the subjects have demonstrated disease progression during or after ≥1 prior standard of care systemic anti-cancer therapy (e.g., chemotherapy, targeted therapy) for metastatic disease. In exemplary aspects, the subject has measurable disease as defined by ≥1 metastatic liver lesion that can be accurately and serially measured in ≥1 dimension and for which the longest diameter is ≥1 cm as measured by multiphase CT scan or magnetic resonance imaging (MRI). In exemplary instances, the subject has ≥1 injectable metastatic liver lesion without necrosis ≥1 cm in longest diameter or ≥1 metastatic liver lesion with necrosis where the longest diameter of the necrotic region subtracted from longest diameter of the lesion is ≥1 cm. In some instances, the subject has an Eastern Cooperative Oncology Group (ECOG) performance status of 0 or 1 and/or a life expectancy ≥5 months. In some aspects, the subject meets one or more of the hematological, renal, hepatic, or coagulation criteria in Table 3:

TABLE 3

| | |
|---|---|
| Hematological | ANC ~1500/mm³ (10⁹/L) without granulocyte colony- |
| | stimulating factor support within 2 weeks prior to enrollment |
| | white blood cell counts (WBC) >2500/μL(10⁹/L) |
| | lymphocyte count ≥500/μL(10⁹/L) |
| | platelet count ~100,000/mm³ (10⁹/L) |
| | hemoglobin ~9 g/dL (90 g/L) (without need for hematopoietic growth |
| | factor or transfusion support within 2 weeks prior to enrollment) |
| Renal | serum creatinine ~1.5 × upper limit of normal (ULN), OR |
| | creatinine clearance ≥60 mL/min/1.73m² for a subject with |
| | serum creatinine levels >1.5 × ULN. (Note creatinine clearance |
| | need not be determined if the baseline serum creatinine is ≤1.5 × |
| | ULN. Creatinine clearance should be determined per |
| | institutional standards) |
| Hepatic | serum albumin ≥2.5 g/dL |
| | serum total bilirubin ~1.5 × ULN |
| | aspartate aminotransferase (AST) ~2.5 × ULN |
| | alanine aminotransferase (ALT) ~2.5 × ULN |
| | alkaline phosphatase (ALP) ≤2.5 × ULN |
| Coagulation | prothrombin time (PT) or international normalization ratio (INR) ≤1.5 × |
| | ULN |
| | partial thromboplastin time (PTT) or activated PTT (aPTT) ≤1.5 × ULN |

Kits

The present disclosure also provides kits comprising an oncolytic virus and an anti-PD-L1 antibody. In exemplary aspects, the oncolytic virus is packaged separately from the anti-PD-L1 antibody. For example, the kit comprises a first container housing the oncolytic virus and a second container housing the anti-PD-L1 antibody. In exemplary aspects, the first container and the second container are provided together, e.g., packaged into one box or larger container. In alternative aspects, the first container is provided to the user separately from the second container. In alternative aspects, the oncolytic virus is packaged together with the anti-PD-L1 antibody. For example, the kit comprises a single container comprising both the oncolytic virus and the anti-PD-L1 antibody. In exemplary aspects, the oncolytic virus is talimogene laherparepvec and the anti-PD-L1 antibody is atezolizumab.

In exemplary aspects, each of the oncolytic virus and the anti-PD-L1 antibody is provided as a unit dose. For purposes herein “unit dose” refers to a discrete amount dispersed in a suitable carrier. In exemplary aspects, the unit dose is the amount sufficient to provide a subject with a desired effect, e.g., reduction of tumor burden, treatment of triple negative breast cancer or colorectal cancer with liver metastases. In exemplary aspects, the oncolytic virus is provided as a sterile frozen suspension. In exemplary aspects, the anti-PD-L1 antibody is provided as a refrigerated solution. In exemplary aspects, the kit comprises several unit doses, e.g., half year or year supply of unit doses, optionally, each of which is individually packaged or otherwise separated from other unit doses. In some embodiments, the components of the kit/unit dose are packaged with instructions for administration to a subject. In some embodiments, the kit comprises one or more devices for administration to a patient, e.g., a needle and syringe, an infusion bag, and the like. In some aspects, the oncolytic virus and/or the anti-PD-L1 antibody is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein.

In exemplary embodiments, the presently disclosed method is as described below:

1. A method of treating a subject with triple negative breast cancer or colorectal cancer, comprising administering to the subject a combination of an oncolytic virus and an anti-PD-L1 antibody, wherein the oncolytic virus is administered to the subject at an initial dose followed by a second dose, wherein the initial dose is lower than the second dose.

2. The method according to embodiment 1, wherein said oncolytic virus is administered intratumorally.

3. A method of treating a subject with triple negative breast cancer with liver metastases or with colorectal cancer with liver metastases, comprising administering to the subject a combination of an oncolytic virus and an anti-PD-L1 antibody, wherein the oncolytic virus is intrahepatically administered to the subject.

4. The method of embodiment 3, wherein the oncolytic virus is administered to one or more injectable liver lesions in the subject.

5. The method of embodiment 3 or 4, wherein the oncolytic virus is administered into liver metastases by imaged guided injection via ultrasound or computerized tomography into injectable liver lesions.

6. The method of any one of embodiments 3 to 5, wherein the oncolytic virus is administered to the subject at an initial dose followed by a second dose, wherein the initial dose is lower than the second dose.

7. The method of embodiment 1, 2, or 6, wherein the second dose of the oncolytic virus is administered about 27 days to about 31 days after the initial dose.

8. The method of embodiment 7, wherein at least one subsequent dose of the oncolytic virus is administered after the second dose.

9. The method of embodiment 8, wherein at least one subsequent dose of the oncolytic virus is administered about 21 days after the second dose.

10. The method of embodiment 9, comprising administering at least two, three, or four subsequent dose(s) of the oncolytic virus about every 21 days after administration of the second dose.

11. The method of any one of embodiments 1 or 6 to 10, wherein the initial dose of the oncolytic virus is not more than 4.0 ml of a solution comprising the oncolytic virus at a concentration of about 10⁶ PFU/ml.

12. The method of any one of embodiments 1 or 6 to 11, wherein the second dose of the oncolytic virus is not more than 4.0 ml of a solution comprising the oncolytic virus at a concentration of about 10⁸ PFU/ml.

13. The method of any one of embodiments 8 to 12, wherein the subsequent dose of the oncolytic virus is not more than 4.0 ml of a solution comprising the oncolytic virus at a concentration of about 10⁸ PFU/ml.

14. The method of any one of the preceding embodiments, wherein the PD-L1 antibody is intravenously administered to the subject.

15. The method of embodiment 14, comprising administering to the subject the PD-L1 antibody over about 45 minutes to about 75 minutes.

16. The method of embodiment 15, further comprising administering a second administration of the PD-L1 antibody.

17. The method of embodiment 16, wherein the second administration occurs over about 20 minutes to about 40 minutes.

18. The method of embodiment 16 or 17, wherein the second administration of the PD-L1 antibody occurs about 21 days to about 24 days after the first administration.

19. The method of embodiment 18, wherein the second administration of the PD-L1 antibody occurs about 21 days after the first administration.

20. The method of any one of embodiments 16 to 19, wherein at least one subsequent administration of the PD-L1 antibody is given to the subject after the second administration.

21. The method of embodiment 20, wherein the subsequent administration of the PD-L1 antibody occurs about 18 days to about 24 days after the second administration.

22. The method of any one of the preceding embodiments, wherein the PD-L1 antibody is administered at a dose of about 1000 mg to about 1500 mg.

23. The method of embodiment 22, wherein the PD-L1 antibody is administered at a dose of about 1150 mg to about 1350 mg, 24. The method of embodiment 23, wherein the PD-L1 antibody is administered at a dose of about 1200 mg.

25. The method of any one of the preceding embodiments, wherein the oncolytic virus is an oncolytic herpes simplex virus (HSV).

26. The method of embodiment 25, wherein the oncolytic HSV is a replication-competent, attenuated HSV-1.

27. The method of embodiment 26, wherein the HSV-1:
lacks a functional ICP34.5 encoding gene;
lacks a functional ICP47 encoding gene; and
comprises a gene encoding human granulocyte macrophage-colony stimulating factor (GM-CSF).

28. The method of any one of the preceding embodiments, wherein the oncolytic virus is talimogene laherparepvec.

29. The method of any one of the preceding embodiments, wherein the PD-L1 antibody is a blocking antibody.

30. The method of any one of the preceding embodiments, wherein the PD-L1 antibody is a humanized antibody.

31. The method of any one of the preceding embodiments, wherein the PD-L1 antibody is an IgG1 antibody.

32. The method of any one of the preceding embodiments, wherein the PD-L1 antibody is monoclonal antibody.

33. The method of any one of the preceding embodiments, wherein the PD-L1 antibody is atezolizumab.

34. A method of treating a subject with triple negative breast cancer or colorectal cancer, comprising administering to the subject a combination of talimogene laherparepvec and atezolizumab, wherein talimogene laherparepvec is administered to the subject at an initial dose followed by a second dose, wherein the initial dose is lower than the second dose.

35. A method of treating a subject with triple negative breast cancer with liver metastases or with colorectal cancer with liver metastases, comprising administering to the subject a combination of talimogene laherparepvec and atezolizumab, wherein talimogene laherparepvec is intrahepatically administered to the subject.

36. A method of treating a subject with triple negative breast cancer or colorectal cancer metastases, comprising administering to the subject a combination of an oncolytic virus and an anti-PD-L1 antibody, wherein the oncolytic virus is administered to the subject at an initial dose followed by a second dose, wherein the initial dose is lower than the second dose.

37. A method of treating a subject with triple negative breast cancer or colorectal cancer metastases, comprising administering to the subject a combination of an oncolytic virus and an anti-PD-L1 antibody, wherein the oncolytic virus is intrahepatically administered to the subject.

38. A method of treating a subject with triple negative breast cancer or colorectal cancer metastases, comprising administering to the subject a combination of talimogene laherparepvec and atezolizumab, wherein talimogene laherparepvec is administered to the subject at an initial dose followed by a second dose, wherein the initial dose is lower than the second dose.

39. A method of treating a subject with triple negative breast cancer or colorectal cancer metastases, comprising administering to the subject a combination of talimogene laherparepvec and atezolizumab, wherein talimogene laherparepvec is intrahepatically administered to the subject.

40. The method of any one of the preceding embodiments, wherein the liver lesion is non-resectable.

The following examples are given merely to illustrate the present present disclosure and not in any way to limit its scope.

### EXAMPLES

### Example 1

This example demonstrates an exemplary method of treating patients with triple negative breast cancer or colorectal cancer with liver metastases.

A Phase 1b study is carried out to confirm the safety, as assessed by incidence of dose limiting toxicities (DLTs), of intrahepatic injection of talimogene laherparepvec into liver metastases in combination with intravenously administered atezolizumab separately in subjects with triple-negative breast cancer or with colorectal cancer. The study is also carried out to evaluate the efficacy of talimogene laherparepvec in combination with atezolizumab separately in subjects with metastatic triple negative breast cancer or with metastatic colorectal cancer with liver metastases, as assessed by Objective response rate (ORR), best overall response (BOR), duration of response (DOR), lesion level responses in injected and uninjected tumor lesions (overall, hepatic, nonhepatic) disease control rate (DCR), durable response rate (DRR), progression-free survival (PFS), overall survival (OS) by cohort (triple-negative breast cancer and colorectal cancer). Furthermore, the study is carried out to confirm the safety and tolerability of intrahepatic injection of talimogene laherparepvec into liver metastases in combination with intravenously administered atezolizumab separately in subjects with triple-negative breast cancer and colorectal cancer.

The phase lb, multicenter, open-label study is designed to confirm the safety of intrahepatic injection of talimogene laherparepvec in combination with intravenously administered atezolizumab in subjects with triple-negative breast cancer and colorectal cancer with liver metastases. Talimogene laherparepvec is injected intrahepatically in combination with intravenous atezolizumab to approximately 36 subjects in 2 parallel cohorts. Cohort 1 comprises subjects with triple negative breast cancer with liver metastases (n =18). Cohort 2 comprises subjects with colorectal cancer with unresectable liver metastases (n=18). The DLT evaluation period is 2 cycles from the initial dose of talimogene laherparepvec in combination with atezolizumab. DLTs are evaluated based on the first 18 DLT-evaluable subjects in each cohort separately. A Dose Level Review Team (DLRT) is to review the safety data to evaluate possible drug effects and DLT. To be evaluable for a DLT, subjects are to have the opportunity to be on treatment for at least 2 cycles from the initial dose of study treatment and receive at least 2 doses of talimogene laherparepvec and 2 doses of atezolizumab in combination, or have a DLT during the DLT evaluation period. Subjects are replaced if they are not evaluable for DLT in order to obtain 18 DLT-evaluable subjects. There is 1 safety interim analysis after the first 4 to 6 subjects on the study have been enrolled, and a final analysis after 18 subjects have been enrolled in each cohort. Enrollment in both cohorts is suspended during the first safety interim analysis. At the discretion of the DLRT, additional safety analyses are conducted as warranted. Treatment continues until a subject experiences a DLT (during the DLT evaluation period), has a complete response (CR), has need for an alternative anticancer therapy, or experiences a safety concern. In addition, treatment discontinues for talimogene laherparepvec if the subject has no injectable lesions, upon confirmed progressive disease (PD) per modified immune related response criteria Response Evaluation Criteria in Solid Tumors (irRC-RECIST) or rapid clinical deterioration. Atezolizumab is discontinued upon symptomatic disease progression. All subjects are to complete a safety follow-up visit approximately 30 (+7) days after the last dose of study treatment. After the safety follow-up visit, all subjects are to enter the long-term follow-up. Subjects are followed for survival, subsequent anticancer therapies and treatment-related adverse events every 12 weeks (±28 days) for approximately 24 months after the last subject is enrolled.

Approximately 36 subjects are enrolled (18 subjects in each cohort). The study subjects are age >18 years and have a diagnosis of triple negative breast cancer or colorectal cancer with liver metastases. Subjects have disease progression during or after >1 prior standard of care systemic anti-cancer therapy for metastatic disease. Subjects have measurable liver lesions that are suitable for injection. Subjects have Eastern Cooperative Oncology Group (ECOG) performance status of 0 or 1, adequate organ function and life expectancy >5 months. Female subjects of childbearing potential have a negative serum pregnancy test. Subjects are excluded if they are candidates for hepatic surgery or locoregional therapy of liver metastases with curative intent, or if more than one-third of the liver is estimated to be involved with metastases or if they have macroscopic intravascular invasion into the main portal vein, hepatic vein, or vena cava. Subjects are ineligible if they are receiving or have received liver metastatic-directed therapy (eg, radiation, ablation, embolization), hepatic surgery, antibody-based therapy, or immunotherapy <4 weeks prior to enrollment. Subjects with a history of malignancy (other than the current malignancy) within the past 5 years are excluded with some exceptions. Subjects with active or untreated central nervous system (CNS) metastases, presence of leptomeningeal disease or spinal cord compression are excluded. Subjects with symptomatic autoimmune disease or who are immunosuppressed are excluded. Subjects with active herpetic skin lesions or prior complications of herpetic infection (eg, herpetic keratitis or encephalitis), or who require intermittent and chronic systemic treatment with an antiherpetic drug (other than intermittent topical use), are not eligible for the study. Subjects receiving concomitant treatment with warfarin are not eligible for the study.

Talimogene laherparepvec is supplied as a sterile frozen liquid in a single-use vial. Each vial contains a minimum of 1.0 mL talimogene laherparepvec at either 10⁶ plaque-forming unit (PFU)/mL or 10⁸ PFU/ concentrations. The first cycle of talimogene laherparepvec is 21 (±3) days. Subsequent cycles of talimogene laherparepvec are 21 days. On cycle 1, day 1, the first dose of talimogene laherparepvec is up to 4.0 mL of 10⁶ PFU/mL. During the second cycle, talimogene laherparepvec is administered up to 4.0 mL of 10⁸ PFU/mL at week 4 of the study (±3 days). During subsequent cycles, talimogene laherparepvec is administered up to 4.0 mL of 10⁸ PFU/mL every 21 days (±3 days) thereafter. The maximum volume of talimogene laherparepvec to be administered at any dose is 4.0 mL for any individual tumor lesion or for all tumor lesions combined. Talimogene laherparepvec is administered by imaged guided injection (either ultrasound or computerized tomography [CT]) into injectable liver lesions. After 3 cycles, injection of non-hepatic lesions is permitted if volume remains after injecting liver lesions. After 6 cycles of intrahepatic talimogene laherparepvec are administered, there is an investigator option to continue talimogene laherparepvec injections for up to an additional 6 cycles (for a maximum of 12 total cycles of talimogene laherparepvec). During this additional dosing period (cycles 7 to 12), talimogene laherparepvec may be administered by intralesional injection to liver metastases or cutaneous, subcutaneous, and nodal tumor lesions, or both. For cycles 7 to 12, liver lesions do not need to be prioritized.

Atezolizumab is supplied as a single-use, 20-cc Pharmacopeia (USP)/European Pharmacopoeia (Ph. Eur.) type 1 glass vial as a colorless-to-slightly-yellow, sterile, preservative-free clear liquid solution intended for intravenous administration. The vial is designed to deliver 20 mL (1200 mg) of atezolizumab solution but may contain more than the stated volume to enable delivery of the entire 20 mL volume. The first cycle of atezolizumab is 21 (±3) days. Subsequent cycles of atezolizumab are 21 (±3) days. Atezolizumab is administered intravenously at a dose of 1200 mg. Administration of atezolizumab is performed in a setting with emergency medical facilities and staff who are trained to monitor for and respond to medical emergencies. The initial dose of atezolizumab (day 1, cycle 1) is delivered over 60 (±15) minutes. If the first infusion is tolerated without infusion-associated adverse events, the second infusion may be delivered over 30 (±10) minutes. If the 30-minute infusion is well tolerated, all subsequent infusions may be delivered over 30 (±10) minutes. The subject's vital signs are determined up to 60 minutes before each atezolizumab infusion. Vital signs are also obtained during or after the atezolizumab infusion if clinically indicated.

When possible, atezolizumab is administered prior to talimogene laherparepvec. Talimogene laherparepvec is administered within 23 hours of atezolizumab administration. If atezolizumab is administered after talimogene laherparepvec, it is not administered until the talimogene laherparepvec observation period has ended. The date of the first dose of investigational products is defined as day 1 (week 1). All subsequent doses and study visits are scheduled based on the day 1 date. Investigational product administration begins as soon as possible after enrollment but no later than 5 days after enrollment. Investigational products are to be administered after all other study procedures are completed, during each visit that it is required. It is recommended that dosing occur on the same day of the week (e.g., if first dose is administered on Monday, all subsequent doses are administered on a Monday), however a ±3 day dosing and study procedure window is allowed unless specified otherwise.

Clinical measurements of cutaneous, subcutaneous, and palpable nodal tumor lesions by caliper are measured at baseline as well as at subsequent tumor assessments. Skin lesions are documented by color photography and the photo is to include a ruler to estimate the size of the lesion. Clinically applicable tumor measurements include but are not limited to cancer antigen 27.29 (CA 27.29), cancer antigen 15-3 (CA 15-3), carcinoembryonic antigen (CEA), and cancer antigen 19-9 (CA 19-9). Tumor marker measurements are performed according to institutional guidelines and availability. Tumor marker measurement is required to confirm CR if screening level of tumor marker was above the ULN and criteria for CR per modified irRC-RECIST guidelines are met. Blood for biomarker analysis are collected prior to talimogene laherparepvec administration and approximately 4 hours (±30 minutes) following talimogene laherparepvec administration.

All known sites of disease are to be documented at screening and reassessed at each subsequent tumor evaluation.

Screening assessments are to include CT scans (with oral/IV contrast unless contraindicated) or MRI of the chest, abdomen, and pelvis. A spiral CT scan of the chest can be obtained. An MRI or a non-contrast CT scan of the chest, abdomen, and pelvis can be used in subjects for whom CT scans with contrast are contraindicated (ie, subjects with contrast allergy or impaired renal clearance).

A CT (with contrast) or MRI scan of the brain is done at screening to evaluate for the presence of CNS metastasis in subjects with triple negative breast cancer. An MRI scan of the brain is taken to confirm or refute the diagnosis of CNS metastases at baseline in the event of an equivocal scan. Subjects with active or untreated CNS metastases are not eligible for this study.

If a CT scan for tumor assessment is performed in a positron emission tomography (PET)/CT scanner, the CT acquisition should be consistent with the standards for a full-contrast diagnostic CT scan.

Bone scans are performed if clinically indicated. At the investigator's discretion, other methods of assessment of measurable disease as per modified irRC-RECIST may be used (Example 2).

The same radiographic procedure used to assess disease sites at screening should be used throughout the study (eg, the same contrast protocol for CT scans). All known sites of disease are documented at screening and re-assessed at each subsequent tumor evaluation. Response is assessed by the investigator using modified irRC-RECIST criteria (Example 2). Assessments are performed by the same evaluator if possible to ensure internal consistency across visits.

Liver tumor biopsies are performed immediately prior to the talimogene laherparepvec administration at weeks 1, 7 and 16.

One injected lesion is biopsied at each time point. Liver tumor biopsies are to be collected and pharmacodynamic changes analyzed to determine the effects on the tumor microenvironment including immune profile and adaptive resistance.

### Example 2

This example describes exemplary methods of measurement of tumor lesions and additional parameters for evaluating efficacy of treatment, particularly in the context of the study described in Example 1.

Computed Tomography Scans (or Magnetic Resonance Imaging):

Computed tomography (CT) scans by contrast-enhanced or spiral scan (or magnetic resonance imaging [MRI] scan) are performed to evaluate tumor response for visceral or nodal/soft tissue disease (including lymph nodes). Measurability of lesions on CT scan is based on the assumption that CT slice thickness is 5 mm or less. MRI can be used to assess disease extent if used throughout the study.

The same method of assessment and the same technique can be used to characterize each identified and reported lesion at baseline and during follow-up. A switch from contrast enhanced CT to noncontrast CT or to MRI (or vice versa) is not to preclude response assessment if, in the judgment of the site radiologist, there is no significant difference in the assessment by changing modalities. This may occur if a subject has developed a medical contraindication to intravenous contrast for CT scans while on trial.

Positron Emission Tomography (PET)/CT Scans:

If a combined PET/CT scan is performed, the CT portion of that exam is not to substitute for the dedicated CT exams. The PET portion of the CT can introduce additional data which may bias the investigator assessment of response if it is not routinely or serially performed. However, if the investigator or the site radiologist documents that the CT performed as part of a PET/CT is of identical diagnostic quality to a diagnostic CT (with intravenous and oral contrast) then the CT portion of the PET/CT can be used for tumor measurements.

Ultrasound:

Ultrasound in some aspects is not to be used as a primary method to assess lesion measurements in response to treatment. If new lesions are identified by ultrasound in the course of the study, confirmation by CT or MRI can be performed.

Clinical Lesion Measurements

Clinical lesions are considered measurable when they are superficial and >10 mm in diameter as assessed using calipers (eg, skin nodules). Skin lesions are documented by color photography and the photo is to include a ruler to estimate the size of the lesion. When lesions are evaluable by both clinical exam and imaging, imaging evaluation should be undertaken.

At baseline, lesions are categorized as measurable or non-measurable according to the following definitions:

Measurability of Tumor Lesions at Baseline Measurable Lesions

Measurable lesions are defined at baseline as lesions that can be accurately measured in at least one dimension (ie, longest diameter for non-nodal lesions and short axis for lymph nodes will be measured and followed) with a minimum size of:

≥10 mm by CT scan (CT scan slice thickness no greater than 5 mm) or MRI

≥10 mm caliper measurement by clinical exam for superficial cutaneous or subcutaneous lesion as measured by caliper

A lymph node must be ≥15 mm in short axis when assessed by CT scan or MRI

Target lesions are not to be chosen from a previously irradiated field unless there has been documented tumor progression in that field prior to enrollment. The distribution of the target lesions should be representative of the subject's overall disease (eg, largest lesions per organ).

Non-Measurable Lesions:

All other lesions, including small lesions (longest diameter <10 mm or pathological lymph nodes with ≥10 mm but <15 mm short axis) and other truly non-measurable lesions are considered non-measurable and characterized as non-target lesions. This can include any measurable lesions beyond the maximum number of 10 total (maximum 5 per organ) at baseline and new measurable lesions that were not chosen as target lesions. Only cancerous lesions are selected as non-measurable lesions and not indeterminate lesions and lesions that could be cancer. Other examples of non-measurable lesions include some bone lesions, leptomeningeal disease, inflammatory breast disease, lymphangitic involvement of the skin or (lymphangitis cutis/pulmonis), and groups of lesions that are small and numerous.

Bone Lesions

Bone scans, PET scans or plain films can be used to confirm the presence or absence of bone lesions.

Osteolytic (lytic) bone lesions or mixed lytic-blastic lesions, with identifiable soft tissue components, that can be evaluated by cross-sectional imaging technique such as CT or MRI can be considered as measurable lesions if the soft tissue component meets the definition of measurability as described above. Only the soft tissue component of the bone lesion is to be measured.

Many osteoblastic (blastic) bone abnormalities can be benign and should not be selected as baseline lesions. An isolated new small blastic lesion may not be selected as a new lesion unless there is demonstrated growth on subsequent scans. Multiple new blastic lesions that are clearly cancerous may be considered for new lesions.

Cystic Lesions

Lesions that meet the criteria for radiographically defined simple cysts are not to be considered as malignant lesions (neither measurable or non-measurable) since they are, by definition, simple cysts.

Cystic lesions thought to represent cystic metastases are to be considered as measurable lesions, if they meet the definition of measurability described above. However, if non-cystic lesions are present in the same subject, these are preferred for selection as target lesions. If a cystic lesion is clearly cancerous and has both cystic and solid components, then the complete lesion is to be measured including both components without excluding the cystic portion of a cystic tumor lesion when measuring.

Lesions with Prior Local Treatment

Tumor lesions situated in a previously irradiated area, or an area subject to other localized therapies (e.g., radiation, ablation, embolization), may not be considered measurable unless there has been demonstrated progression in the lesion.

Baseline Documentation of “Target” and “Non-Target” Lesions

Baseline evaluations are to be used to prospectively identify all sites of disease present as close as possible to the enrollment and never more than 4 weeks before the enrollment date. Sites of disease are to be characterized as either target or non-target lesions.

Baseline Documentation of Target Lesions

Up to 10 target lesions (a maximum of 5 per organ) are to be chosen to measure over the course of therapy. Pathological lymph nodes that are defined as measurable are to meet the criterion of a short axis of ≥15 mm by CT scan in order to be identified as target lesions.

The distribution of these target lesions is to be representative of the subject's overall disease status. Target lesions may be selected on the basis of their size (lesions with longest diameter) and suitability for accurate repeated measurements by imaging techniques. In situations where larger lesions cannot be accurately measured repeatedly (eg, near the diaphragm where respiratory changes may affect measurements), smaller lesions that meet criteria for measurability may be selected instead.

A sum of the diameters (longest for non-nodal lesions, short axis for nodal lesions) for all target lesions is to be calculated and reported as the baseline sum of diameters.

Baseline Documentation of Non-Target Lesions

All other lesions (or sites of disease), including any measurable lesions that were not chosen as target lesions and pathological lymph node with short axis ≥10 mm but <15 mm, should be identified as non-target lesions. Measurable non-target lesions (i.e., lesions in an organ beyond the allowed maximum number of targets that would otherwise qualify as target lesions) should also be recorded and assessed qualitatively over the course of the study. Non-measurable non-target disease measurements are not required, but these lesions are evaluated at each timepoint and will be evaluated as ‘present’, ‘absent’, or in rare cases ‘unequivocal progression’.

Follow-up Assessment of Tumor Lesions

At each subsequent tumor assessment, the sum of diameters of target lesions identified at baseline plus the sum of diameters of up to 10 (maximum 5 per organ) new measurable lesions (for which the longest diameter is ≥10 mm for non-nodal lesions or the short axis is ≥15 mm for nodal lesions) are added together to provide the total tumor burden. If more than 10 new measurable lesions total (or 5 per organ) are present, the new measurable lesions are selected on the basis of their size and suitability for accurate repeated measurements by imaging techniques (CT or MRI). If there are lesions beyond the new measurable lesion limit during the course of the study for one subject, the additional lesions are considered new non-measurable lesions.

Tumor Burden=sum of diameter of target lesions +sum of diameter of up to 10 (maximum 5 per organ) new, measurable lesions.

Non-target disease measurements are not required and these lesions are followed as “present”, “absent”, or “unequivocal progression”.

For non-nodal target lesions that become too small to measure, a value of 5 mm is assigned. If the non-nodal lesion subsequently increases in size to greater than or equal to 5 mm in one dimension, its true size is recorded. If an actual measurement is able to be provided, this is recorded even if it is <5 mm If it is in the opinion of the radiologist that the non-nodal lesion has likely disappeared, the measurement is recorded as “0 mm”. Nodal disease should generally have the actual short axis measurement recorded even if the nodes regress to below 10 mm on study.

Response Evaluation

Evaluation of Objective Response

The subject response is assessed based on tumor burden (the sum of diameters of target lesions plus the sum of up to 10 [maximum 5 per organ] new measurable lesions), and, in the case of complete response (CR), the presence of any non-target and/or new non-measurable lesions. The overall response is derived from timepoint response assessments as described in Table 4 and Table 5.

TABLE 4
Definition of Measurable Tumor Response (Baseline
Target and New, Measurable Lesions)

| | |
|---|---|
| Complete | Disappearance of all lesions (whether measurable or not and |
| Response | whether baseline or new) and confirmation by a repeat, |
| (CR): | consecutive assessment no less than 4 weeks from the date first |
| | documented. Any pathological lymph nodes (whether target or |
| | non-target) must have reduction in short axis to <10 mm. |
| | If tumor markers are initially above the upper limit of normal, |
| | they must normalize to be considered CR. |
| Partial | Decrease in tumor burden* ≥30% relative to baseline |
| Response | confirmed by a consecutive assessment at least 4 weeks (28 |
| (PR): | days) after first documentation |
| Progressive | Increase in tumor burden* ≥20% and at least 5 mm absolute |
| Disease | increase relative to nadir (minimum recorded tumor burden) |
| (PD): | confirmation by a repeat, consecutive assessment no less than 4 |
| | weeks (28 days) from the date first documented PD. |
| Stable | Neither sufficient shrinkage to qualify for CR or PR nor |
| Disease | sufficient increase to qualify for PD. |
| (SD): | |
| Unable to | Any lesion present at baseline which was not assessed or was |
| Evaluate | unable to be evaluated leading to an inability to determine the |
| (UE): | status of that particular tumor for that time point. |
| Not Applicable | No target lesions were identified at baseline |
| (NA) | |

*Tumor Burden = sum of diameter of target lesions + sum of diameter of up to 10 (maximum 5 per organ) new, measurable lesions.
Diameters used:
For nodal disease, shortest axis
For non-nodal disease, longest diameters

TABLE 5
Matrix for Determining the Overall
Response at Each Assessment Point

| Measurable Response | Non-target | Overall Response |
|---|---|---|
| Target and new, measurable | (nonmeasurable and new | Using |
| lesions (tumor burden)^{a}, % | nonmeasureable) | irRC-RECIST |
| ↓100^{b} | Absent/NA^{c} | CR^{d} |
| ↓100 | Present | PR^{d} |
| ↓100 | Unequivocal progression | PR^{d} |
| ↓≥30 | Absent/Present NA^{c} | PR^{d} |
| ↓≥30 | Unequivocal progression | PR^{d} |
| ↓<30 to ↑<20 | Absent/Present/NA^{c} | SD |
| ↓<30 to ↑<20 | Unequivocal progression | SD |
| ↑≥20^{e} | Any | PDb,^{d} |
| UE | Any | UE |
| ND | Any | UE |
| NA^{f} | Any | UE |

CR = complete response; irRC-RECIST = immune-related response criteria Response Evaluation Criteria in Solid Tumors; NA = not applicable; PD = progressive disease; PR = partial response; SD = stable disease; UE = unevaluable
^{a}Disease relative to baseline, including new measurable lesions only (>10 mm).
^{b}Disappearance of all non-lymph node lesions and all lymph nodes <10 mm in short axis would also be CR even if lymph node measurements prevent 100% tumor burden reduction.
^{c}No non-target lesions identified at baseline.
^{d}Assuming response (CR or PR) or progression are confirmed by a second, consecutive assessment at least 4 weeks (28 days) apart.
^{e}In addition to relative increase of ≥20%, the tumor burden must also demonstrate an absolute increase of ≥5 mm from nadir for PD.
^{f}No target lesions identified at baseline. When a subject has only non-measurable disease (ie, no target lesions identified at baseline) the response will be unevaluable.

Determination of BOR is based on changes in total tumor burden from the baseline (nadir, for PD) tumor assessment, regardless of any initial increase in baseline lesions or the appearance of new lesions.

Subjects are considered to have PR or SD even if new lesions were present, as long as they met the respective thresholds of response as described in Table 5.

The best overall response for an unconfirmed CR or PR will be SD, and it will be UE if the last overall response is PD in the absence of consecutive confirmation or clinical deterioration. A best overall response of SD requires a visit response of SD or better no earlier than 63 days after the start of treatment; otherwise the overall response will be UE.

Confirmation of Response (CR or PR)

To be assigned a BOR of CR or PR, a corresponding overall visit response of CR or PR is confirmed by consecutive repeat assessments performed no less than 4 weeks (28 days) after the criteria for response are first met.

In some circumstances it may be difficult to distinguish residual disease from normal tissue. When the evaluation of CR depends on this determination, it is recommended that the residual lesion be investigated (ie, biopsy) to confirm the CR status.

Confirmation of Disease Progression

If a subject is classified as having PD at a post baseline tumor assessment, then confirmation of PD by a second assessment ≥4 weeks (28 days) later in the absence of rapid clinical deterioration (eg, rapid decline in performance status) or symptomatic disease requiring rapid initiation of alternative systemic anti-cancer therapy is performed. The definition of confirmation of progression represents a ≥20% and at least 5 mm absolute increase in the total tumor burden (ie, the sum of diameters of target lesions plus up to 10 [maximum 5 per organ] new measurable lesions) compared to the nadir at 2 consecutive time-points at least 4 weeks (28 days) apart (with the date of progression considered to be the time of the initial evaluation showing PD).

Subjects with a global deterioration of health status requiring discontinuation of treatment without objective evidence of PD at the time may have the reason for treatment discontinuation specified. Every effort is made to document the objective progression even after discontinuation of treatment.

Subjects who have had a procedure to completely/partially resect a lesion are evaluated as follows:

The procedure itself and all post-procedure lesion assessments should always be recorded in the CRF. A completely resected lesion is assigned a default code of 0 mm (for target lesions) or “absent” (for non-target lesions). A partially resected lesion is assigned its measurement post-procedure (for target lesions) or “present” (for non-target lesions). If the resected lesion contained no cancer under pathology evaluation, subsequent tumor assessments post-procedure is used for tumor burden calculations and/or determination of response. If the resected lesion contained cancer or pathology results were unknown, the recorded tumor assessments post-procedure may be used for tumor burden calculations, but determination of response will be considered unevaluable (UE) for response except in the case of PD.

If the new tumor burden post-procedure is lower than the nadir before the procedure, then the new nadir is set to the post-procedure tumor burden. Otherwise, the previous pre-procedure nadir is retained as the nadir. Subsequent assessments for PD will be determined from the nadir.

Merging Lesions

When two or more target/new measurable lesions merge, the smaller lesion is to have 0 mm recorded for the current and all future assessments, and the larger lesion is to have the longest diameter of the merged lesion recorded for the current assessment and be followed for future assessments. When two or more non-target/new non-measurable lesions merge, the smaller lesion is recorded as absent for the current and all future assessments, and the larger lesion is recorded as present for the current assessment and followed for future assessments. If a target/new measurable lesion and a non-target/new non-measurable lesion merge, the non-target/new non-measurable lesion is absent for the current and all future assessments while the target lesion/new measurable lesion is to include both merged lesions for recording measurements.

Separating Lesions

When a target/new measurable lesion splits into 2 or more lesions, the largest measurable part of the split lesion is considered to be the previously recorded target/new measurable lesion with measurements provided for the current assessment and followed for future assessments. The dimensions of the split parts is still considered measurable. Any new lesions that result from separating is documented as lesions that were generated by separating and not truly new lesions. When a non-target lesion splits into 2 or more lesions, the split parts remain non-target lesions for the duration of the study.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of treating a subject with triple negative breast cancer or colorectal cancer, comprising administering to the subject a combination of an oncolytic virus and an anti-PD-L1 antibody, wherein the oncolytic virus is administered to the subject at an initial dose followed by a second dose, wherein the initial dose is lower than the second dose.

2. The method according to claim 1, wherein said oncolytic virus is administered intratumorally.

3. A method of treating a subject with triple negative breast cancer with liver metastases or with colorectal cancer with liver metastases, comprising administering to the subject a combination of an oncolytic virus and an anti-PD-L1 antibody, wherein the oncolytic virus is intrahepatically administered to the subject.

4. The method of claim 3, wherein the oncolytic virus is administered to one or more injectable liver lesions in the subject.

5. The method of claim 3 or 4, wherein the oncolytic virus is administered into liver metastases by imaged guided injection via ultrasound or computerized tomography into injectable liver lesions.

6. The method of any one of claims 3 to 5, wherein the oncolytic virus is administered to the subject at an initial dose followed by a second dose, wherein the initial dose is lower than the second dose.

7. The method of any one of the preceding claims, wherein the PD-L1 antibody is intravenously administered to the subject.

8. The method of any one of the preceding claims, wherein the oncolytic virus is an oncolytic herpes simplex virus (HSV).

9. The method of claim 8, wherein the oncolytic HSV is a replication-competent, attenuated HSV-1.

10. The method of claim 9, wherein the HSV-1:
lacks a functional ICP34.5 encoding gene;
lacks a functional ICP47 encoding gene; and
comprises a gene encoding human granulocyte macrophage-colony stimulating factor (GM-CSF).

11. The method of any one of the preceding claims, wherein the oncolytic virus is talimogene laherparepvec.

12. The method of any one of the preceding claims, wherein the PD-L1 antibody is a blocking antibody.

13. The method of any one of the preceding claims, wherein the PD-L1 antibody is a humanized antibody.

14. The method of any one of the preceding claims, wherein the PD-L1 antibody is an IgG1 antibody.

15. The method of any one of the preceding claims, wherein the PD-L1 antibody is monoclonal antibody.

16. The method of any one of the preceding claims, wherein the PD-L1 antibody is atezolizumab.

17. A method of treating a subject with triple negative breast cancer or colorectal cancer metastases, comprising administering to the subject a combination of an oncolytic virus and an anti-PD-L1 antibody, wherein the oncolytic virus is administered to the subject at an initial dose followed by a second dose, wherein the initial dose is lower than the second dose.

18. The method of claim 17, comprising administering to the subject a combination of talimogene laherparepvec and atezolizumab, wherein talimogene laherparepvec is administered to the subject at an initial dose followed by a second dose, wherein the initial dose is lower than the second dose.

19. A method of treating a subject with triple negative breast cancer or colorectal cancer metastases, comprising administering to the subject a combination of an oncolytic virus and an anti-PD-L1 antibody, wherein the oncolytic virus is intrahepatically administered to the subject.

20. The method of claim 19, comprising administering to the subject a combination of talimogene laherparepvec and atezolizumab, wherein talimogene laherparepvec is intrahepatically administered to the subject.

21. The method of any one of the preceding claims, wherein the liver lesion is non-resectable.


SEQUENCE LISTING (15 sequences)

SEQ ID NO: 1 - protein, 290 aa, Homo Sapiens
  ref <308>: DATABASE ACCESSION NUMBER: UniProt
  ref <309>: DATABASE ENTRY DATE: 2000-10-01
  ref <313>: RELEVANT RESIDUES IN SEQ ID NO: (1)..(290)
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 10 aa, Homo Sapiens
GFTFSDSWIH

SEQ ID NO: 3 - protein, 18 aa, Homo Sapiens
AWISPYGGSTYYADSVKG

SEQ ID NO: 4 - protein, 9 aa, Homo Sapiens
RHWPGGFDY

SEQ ID NO: 5 - protein, 11 aa, Homo Sapiens
RASQDVSTAVA

SEQ ID NO: 6 - protein, 7 aa, Homo Sapiens
SASFLYS

SEQ ID NO: 7 - protein, 9 aa, Homo Sapiens
QQYLYHPAT

SEQ ID NO: 8 - protein, 118 aa, Homo Sapiens [sequence omitted]

SEQ ID NO: 9 - protein, 108 aa, Homo Sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 10 - protein, 447 aa, Homo Sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Homo Sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 449 aa, Homo Sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 216 aa, Homo Sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 14 - protein, 450 aa, Homo Sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 215 aa, Homo Sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC